(19) United States
(12) Patent Application Publication
(10) Pub. No.: US 2026/0307330 A1
(43) Pub. Date: Oct. 8, 2026

(54) MULTI-CHANNELED AND GUIDED INNER CONTROLLING (MAGIC) PRINTHEADS

(71) Applicant: Board of Regents, The University of Texas System, Austin, TX (US)

(72) Inventors: Mohammed Maniruzzaman, Austin, TX (US); Yu Zhang, Austin, TX (US); Faez Alkadi, Austin, TX (US)

(21) Appl. No.: 18/998,355

(22) PCT Filed: Jul. 27, 2023
(86) PCT No.: PCT/US2023/028884

§ 371 Date: Jan. 24, 2025

Related U.S. Application Data
(60) Provisional application No. 63/392,795, filed on Jul. 27, 2022.

(51) Int. Cl. B29C 64/209; B29C 64/106; B29C 64/314; B33Y 10/00; B33Y 30/00; B33Y 40/10

(52) U.S. Cl. CPC B29C 64/209; B29C 64/106; B29C 64/314; B33Y 10/00; B33Y 30/00; B33Y 40/10

(57) ABSTRACT

Described are techniques, systems, and methods including those employing multi-channel controlled pneumatic printheads which contain a linker (e.g. Luer lock), a designed functional system (e.g., a microfluidic system, coaxial system, bi/tri-axial, or a mixing system), and one or more outlets (e.g., nozzle part or output linker). The printheads are useful for pneumatic, pressure assisted, extrusion-based 3D printing and emulsion/mixture evaporation for fabricating particles that may encapsulate an active pharmaceutical ingredient or live cells into a biocompatible polymer.

### CROSS-REFERENCE TO RELATED APPLICATIONS

This application claims the benefit of and priority to U.S. Provisional Application No. 63/392,795, filed on Jul. 27, 2022, which is hereby incorporated by reference in its entirety.

### STATEMENT REGARDING FEDERALLY SPONSORED RESEARCH OR DEVELOPMENT

This invention was made with government support under Grant no. R01 FD007456 awarded by the National Institutes of Health. The government has certain rights in the invention.

### FIELD

This invention is in the field of printing material, such as particles (e.g., microparticles and nanoparticles) or hydrogels (gelations), and delivery of live cells, biologics, or active pharmaceutical ingredients (drugs) by microparticles or nanoparticles. This invention relates generally to devices, systems, and methods for extrusion-based printing of microparticles, nanoparticles, microparticle formulations, nanoparticle formulations, hydrogels, or the like.

### SUMMARY

Microparticles and nanoparticles have attracted worldwide research interests and have emerged as a powerful tool for the delivery of pharmaceutical reagents. The effectiveness and function of biodegradable microparticles depend on several physiochemical properties, including size, surface charge, shape, as well as hydrophobicity, and hydrophilicity. Formulated microparticles allow the encapsulation of a variety of agents, including proteins, plasmid DNA, lipophilic and hydrophilic drugs. Moreover, fabricated microparticles are suitable for many administration routes, such as inhalation, injection, and oral delivery. Different ligands and antibodies can also be attached to the microparticle surface for targeted drug delivery. In addition, PEGylated microparticles with stealth properties are developed to increase the circulation time in vivo for improved therapeutic efficiency.

The present disclosure provides printheads with example features that are designed to control various parameters for fabricating microparticles and/or nanoparticles of various target sizes and functionalities. In some examples, multi-channel controlled pneumatic printheads which contain linkers (e.g., Luer locks), designed functional systems (such as microfluid systems, coaxial systems, biaxial systems, reactors, mixers, stirrers, etc.), and output ports (e.g., multi-nozzles, multi-channels) are provided.

In examples, the linker parts are useful to connect a printhead to a printer, such as a bioprinter including one or multiple pneumatically controlled fluid reservoirs. The linkers can include various linking configurations to link with different printer systems and printheads, such as Luer locks, Luer slips, or the like.

In examples, the designed functional system can comprise a microfluidic system that includes a structure having a controlled fluid path length, controlled fluid path size, and/or controlled fluid path cross-sectional shape, which may vary in two dimensions or three dimensions. In some examples, the designed functional system can be coupled with multiple fluid pneumatic injectors via the linkers and can include multiple mixing chambers. In some examples, the designed functional system is not limited to microfluidics systems, but can include coaxial systems, reactors, mixing/stirring systems, or the like. In some examples, the designed functional system is referred to as a fluid mixer, which may be configured to or used to achieve complete or partial mixing of components or may be configured to or used to achieve coaxial, biaxial, triaxial, or higher order axial flow of components.

In examples, various output ports may be used. For example, one or more than one nozzles can be coupled to the designed functional system for outputting fluid from the nozzle(s). Various sizes, cross-sections, shapes, lengths, or the like of the nozzles can be configured so as to achieve output of material from the nozzle according to desired parameters.

In some examples, the printhead is customized and designed using computer-aided design (CAD) software, which can advantageously allow the printheads to be adapted and used in a variety of different commercial printers. For example, the various portions of the printhead can be designed using CAD software and manufactured using various additive manufacture technology, such as 3D laser sintering, stereolithography (SLA) printing, or the like.

The disclosed printheads have advantages for printing nanoparticles, microparticles, and/or hydrogels, such as, but not limited to, proteins, polymers, mRNA, lipids, liposomes, etc. Depending on the material to be printed, nanoparticles and/or microparticles can be prepared using printheads that are designed and/or configured by adjusting the above parameters (e.g., using CAD software) and printed using additive manufacturing.

Additionally, flow simulation software (e.g., SOLIDWORKS® Flow Simulation) can be used to computationally model fluid dynamics and simulate liquid and gas flows for customizing designs for the printheads. Based on the requirements for the different applications, suitable 3D models of printheads can be generated and flow simulations performed to optimize the printhead, with the printheads prepared quickly (e.g., comparing other manufacturing processes) using additive manufacturing.

In some examples, microparticles and nanoparticles can be prepared using the printheads described herein. For example, the printheads can be used for extrusion-based printing and emulsion/mixture evaporation techniques to fabricate novel polymeric microparticles or nanoparticles, such as comprising polymeric poly(lactide-co-glycolide) (PLGA) or other materials, which may be optionally biocompatible or biodegradable. PLGA is an example biocompatible and biodegradable FDA-approved copolymer, which can be hydrolyzed into lactic and glycolic acid monomers.

In an aspect, methods are described herein, such as methods for preparing particles (e.g., microparticles and/or nanoparticles). In some examples, a method of this aspect comprises providing a first component to a first linker of a plurality of linkers of a printhead; providing a second component to a second linker of the plurality of linkers of the printhead; contacting the first component and the second component in a fluid mixer of the printhead; forming a mixture of the first component and the second component in the printhead; and printing or flowing the mixture from the printhead through one or more outlets. In some examples, the first component comprises a polymer, a non-polymeric excipient or carrier, and/or an active pharmaceutical ingredient. In some examples, the second component comprises a solvent. In some examples, additional components can be provided to additional linkers of the plurality of linkers, such that the additional components are mixed with the first and second component in the fluid mixer. Printing may subject the mixture to shear forces that separate the mixture into a plurality of droplets including particles.

Contacting the first component and the second component may comprise providing the first component to an inlet of a fluid mixer in the printhead and providing the second component to the same or a different inlet of the fluid mixer in the printhead. The fluid mixer may comprise a coaxial or other arrangement of a first fluidic channel in fluid communication with a first linker and a second fluid channel in fluid communication with a second linker, for example. Optionally, the first component is provided to the first linker, and the second component is provided to the second linker, which can direct the components to the fluid mixer. In some examples, the fluid mixer may comprise a stirrer. Optionally, an electric field or a magnetic field may be used for activating the stirrer. The stirrer may optionally rotate or be rotatable about an axis parallel to a flow of the mixture (e.g., through one or more of the outlets) or one or more components, or about an axis perpendicular to a flow of the mixture or one or more of the components, or about an axis with any other orientation relative to the flow of the mixture or one or more of the components. The fluid mixer may optionally comprise a mixing architecture characterized by an “S” shape, a “Y” shape, or a helix shape. Again, one or more additional linkers can be included in the printhead to allow for mixing of 3 or more components in the fluid mixer.

Optionally, the particles and/or droplets prepared according to this aspect may be subjected to further processing. For example, methods of this aspect may further comprise subjecting the droplets to evaporation conditions to evaporate the solvent from the droplets and leave the particles. Methods of this aspect may further comprise washing the plurality of particles, for example. Methods of this aspect may further comprise lyophilizing the plurality of droplets or the particles.

Other components may be included in the mixture. For example, the mixture may comprise or further comprise one or more of a cosolvent, a surfactant, a preservative, live cells, cellular components, an additional active ingredient, a salt, a preservative, a protein, a peptide, an amino acid, or a nucleic acid component. The mixture may be a homogeneous mixture or a non-homogeneous mixture. In some examples, a non-homogeneous mixture may be desirable so as to prevent complete mixing of the first component and a second component, such as to form a coaxial feed of the first component around or adjacent to the second component.

The methods described herein may generally be useful for preparing particles containing any desirable active ingredient. Without limitation, example active ingredients may comprise a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or mega-nucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell. In some examples, a weight ratio of the active pharmaceutical ingredient to the polymer or the non-polymeric excipient in the mixture is from 1:8 to 1:15, such as from 1:8 to 1:9, from 1:9 to 1:10, from 1:10 to 1:11, from 1:11 to 1:12, from 1:12 to 1:13, from 1:13 to 1:14, or from 1:14 to 1:15.

For particles comprising a polymer, the polymer may be a biodegradable polymer. Example biodegradable polymers include, but are not limited to, poly(lactide-co-glycolide), polylactide (PLA), polyglycolide (PGA), polycaprolactone (PCL), Pluronic F127, sodium alginate, hyaluronic acid, chitosan, cyclodextrin, dextran, agarose, gelatin, albumin, collagen, fibroin, lipids, a polyethylene glycol (PEG) derivative, a pharmaceutical grade polymer, poly(hydroxy butyrate), poly(β-malic acid), or poly(L-lysine).

For particles comprising a non-polymeric excipient, the non-polymeric excipient may be a hydrophilic substance or a hydrophobic substance. Example non-polymeric excipients include, but are not limited to, a non-reducing sugar, such as trehalose or sucrose, a polyol, such as mannitol, sorbitol, xylitol, or an amino acid, such as leucine or L-arginine.

The particles can be prepared using any suitable printing parameters and any suitable environmental parameters. In some examples, the printing may occur at ambient conditions (e.g., at atmospheric pressure and at room temperature), though control of the temperature of the mixture may be achieved by including a heat exchanger or other temperature controller (e.g., a coolant jacket, a Peltier cooler, etc.) in the printhead (e.g., at the fluid mixer). Temperatures for collecting the plurality of droplets may correspond to ambient temperature or cryogenic temperatures. For example, collecting the plurality of droplets optionally comprises receiving the plurality of droplets on a surface having a temperature of from about −200° C. to about −78° C. or at room temperature or from about 4° C. to about 50° C. In some examples, the extrusion-based printing method subjects the mixture to a pressure of from 1 kPa to 700 kPa, such as from 1 kPa to 600 kPA, from 1 kPa to 500 kPa, from 1 kPa to 400 kPa, from 1 kPa to 300 kPa, from 1 kPa to 200 kPa, from 1 kPa to 100 kPa, from 1 kPa to 50 kPa, from 1 kPa to 40 kPa, from 1 kPa to 30 kPa, from 1 kPa to 20 kPa, from 1 kPa to 10 kPa, from 10 kPa to 600 kPa, from 10 kPa to 500 kPa, from 10 kPa to 400 kPa, from 10 kPa to 300 kPa, from 10 kPa to 200 kPa, from 10 kPa to 100 kPa, from 10 kPa to 20 kPa, from 20 kPa to 30 kPa, from 30 kPa to 40 kPa, from 40 kPa to 50 kPa, from 50 kPa to 60 kPa, from 60 kPa to 70 kPa, from 70 kPa to 80 kPa, from 80 kPa to 90 kPa, from 90 kPa to 100 kPa, from 110 kPa to 120 kPa, from 120 kPa to 130 kPa, from 130 kPa to 140 kPa, from 140 kPa to 150 kPa, from 150 kPa to 160 kPa, from 160 kPa to 170 kPa, from 170 kPa to 180 kPa, from 180 kPa to 190 kPa, from 190 kPa to 200 kPa, from 200 kPa to 210 kPa, from 210 kPa to 220 kPa, from 220 kPa to 230 kPa, from 230 kPa to 240 kPa, from 240 kPa to 250 kPa, from 250 kPa to 300 kPa, from 300 kPa to 350 kPa, from 350 kPa to 400 kPa, from 400 kPa to 450 kPa, from 450 kPa to 500 kPa, from 500 kPa to 550 kPa, from 550 kPa to 600 kPa, from 600 kPa to 650 kPa, or from 650 kPa to 700 kPa. Optionally, an extrusion pressure of the extrusion-based printing method greater than or about 700 kPa. In some examples, the extrusion-based printing method uses a nozzle having a diameter of from 1 μm to 1000 μm, such as from 1 μm to 10 μm, from 10 μm to 100 μm, from 100 μm to 700 μm, from 300 μm to 700 μm, from 100 μm to 200 μm, from 200 μm to 300 μm, from 300 μm to 400 μm, from 400 μm to 500 μm, from 500 μm to 600 μm, from 600 μm to 700 μm, from 700 μm to 800 μm, from 800 μm to 900 μm, or from 900 μm to 1000 μm. Optionally, a temperature of the mixture during the printing is from about 4° C. to about 50° C., such as from 4° C. to 10° C., from 10° C. to 20° C., from 20° C. to 30° C., from 30° C. to 40° C., or from 40° C. to 50° C. Optionally, printing the mixture comprises receiving the particles or droplets on a surface, wherein the surface has a temperature of about room temperature or less than or about −78° C. or less than or about −180° C.

In another aspect, systems are described herein, such as systems for preparing particles (e.g., microparticles and/or nanoparticles), optionally according to the methods described herein. Optionally, a system of this aspect comprises a printer, such as a bioprinter, which may include one or more pneumatically controlled fluid extruders. In some examples, systems of this aspect comprise a plurality of supply containers for preparing or storing respective components, such as but not limited to components comprising a polymer or a non-polymeric excipient, a solvent, or an active pharmaceutical ingredient; a printhead in fluid communication with the plurality of supply containers, the printhead comprising a plurality of linkers, such as where each linker is in fluid communication with an outlet of one of the plurality of supply containers; a fluid mixer for mixing components from the plurality of linkers into a mixture; and one or more outlets in fluid communication with the fluid mixer. The system may optionally comprise a collection surface for receiving a plurality of droplets of the mixture from the one or more outlets. In examples, the mixture may comprise water, a polymer or a non-polymeric excipient, a solvent, and an active pharmaceutical ingredient. In some examples, the collection surface comprises a sterile vial.

Systems of this aspect can include various components or adjustable parameters to allow for preparing particles, such as according to the methods described herein. In some examples, the collection surface may optionally be cooled to a temperature of from about −200° C. to about −75° C. A system of this aspect may further comprise a cooling or refrigeration system coupled to the collection surface for cooling the collection surface to a temperature of from about −200° C. to about −75° C. Optionally, a system of this aspect may comprise one or more temperature sensors or temperature controllers for monitoring or controlling a temperature of the collection surface. In some examples, the collection surface is a moving or movable or translating or translatable collection surface. Optionally, a system of this aspect may further comprise a translation stage for generating a relative translation between one or more printheads and/or nozzles and the collection surface. In some examples, a system of this aspect may further comprise one or more pressure sensors or pressure controllers for monitoring or controlling an extrusion pressure associated with the printheads and/or nozzles. Optionally, a system of this aspect may further comprise one or more actuators for monitoring or controlling an extrusion speed associated with one or more printheads and/or nozzles.

In some examples, it may be desirable to prepare particles under sterile conditions. Optionally, a system of this aspect may further comprise a housing for maintaining at least one or more printheads and/or nozzles and the collection surface in a sterile environment. Optionally, a system of this aspect may further comprise sterilization equipment positioned to sterilize one or more of the plurality of supply containers, the one or more printheads and/or nozzles, or the collection surface.

In another aspect compositions are provided herein, such as microparticle-based therapeutic compositions. In some examples, a composition may comprise particles having diameters of from 10 nm to 1100 μm; and one or more live cells. In some examples, the particles are attached to surfaces of the one or more live cells. In some examples, the one or more live cells are at least partially encapsulated into the particles. Example live cells include, but are not limited to, live yeast cells, live recombinant yeast cells, live fungal cells, live bacterial cells, live recombinant bacterial cells, live insect cells, live mammalian cells, or live mesenchymal stem cells. Optionally, the particles may be in a lyophilized condition.

Example particles include particles comprising a polymer or a non-polymeric excipient, such as prepared according to various methods described herein or prepared using various systems described herein. Optionally, for particles comprising a polymer, the polymer is a biodegradable polymer selected from the group consisting of poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, fibroin, a polyethylene glycol (PEG) derivative, a pharmaceutical grade polymer, poly(hydroxy butyrate), poly(β-malic acid), or poly(L-lysine). Optionally, for particles comprising a non-polymeric excipient, the non-polymeric excipient is a hydrophilic substance, a hydrophobic substance, a non-reducing sugar, trehalose, sucrose, a polyol, mannitol, sorbitol, xylitol, an amino acid, leucine, or L-arginine.

In some examples, the particles further comprise an active ingredient embedded within or adsorbed to the particles. For example, the active pharmaceutical ingredient may include one or more of a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell.

In another aspect, printheads are described herein, such as printheads for preparing particles, optionally according to the methods described herein. In some examples, the printheads may comprise or correspond to various printheads described herein. The printheads may include a plurality of linkers defining inlets of the printhead; a fluid mixer in fluid communication with the plurality of linkers for mixing components from the inlets into a mixture; and one or more outlets in fluid communication with the fluid mixer.

Optionally, one or more of the plurality of linkers comprises a Luer lock, a Luer slip, or a slip tip. In some examples, the plurality of linkers may be characterized by a diameter of less than or about 5.0 mm. The linkers may be used and/or configured for establishing sealed fluid communication with other devices, such as a bioprinter, pneumatically controlled fluid extruders, supply containers, other printheads or fluid mixers, etc.

In some examples, the fluid mixer may comprise a coaxial or other arrangement of a first fluidic channel in fluid communication with a first linker and a second fluid channel in fluid communication with a second linker. One or more additional fluidic channels in fluid communication with one or more additional linkers may optionally be used. In some examples, the fluid mixer may comprise a stirrer, which may or may not include an electric field or a magnetic field for activating the stirrer. The stirrer may optionally rotate or be rotatable about an axis parallel to a flow of the mixture (e.g., through one or more of the outlets) or one or more components, or about an axis perpendicular to a flow of the mixture or one or more of the components, or about an axis with any other orientation relative to the flow of the mixture or one or more of the components. The fluid mixer may be formed from a simple 2D structure to a complex 2D structure to a 3D structure. The fluid mixer may be any shape with any angle size/channel cross-section and, in examples, may comprise a mixing architecture characterized by a “Y” shape, an “S” shape, or a helix shape. In some cases, mixing architecture can be configured according to any suitable shape, configuration, or scheme for a particular application, such as to achieve a specific amount of mixing and/or to achieve complete and/or partial mixing of two or three or more different components, for example.

In some examples, the one or more outlets may be characterized by a diameter of less than or about 5.0 mm. The one or more outlets may comprise one or more extrusion-based printing nozzles for generating a plurality of droplets of the mixture including particles having diameters of from 10 nm to 1100 μm. In some examples, the one or more outlets may comprise additional linkers, such as for establishing fluid communication with a linker of an additional printhead, such as to provide a mixture of two or more components as an input for the additional printhead.

In another aspect, methods are described herein, such as methods for preparing and/or manufacturing printheads. In some examples, any of the printheads described herein can be prepared or manufactured. In some examples, the printhead comprises a plurality of linkers defining inlets of the printhead; a fluid mixer in fluid communication with the plurality of linkers for mixing components from the inlets into a mixture; and one or more outlets in fluid communication with the fluid mixer.

In some examples, multiple printheads may be linked together, such as where a linker of one printhead is coupled to an outlet of another printhead. Such a configuration may be useful for achieving complex mixing configurations, such as where a coaxial or multi-axial mixture flow of components is achieved by a first printhead and where the mixture flow from the first printhead is further mixed with additional components, such as in a further coaxial or multi-axial mixture flow.

In some examples, manufacturing the printhead may comprise an additive manufacturing process (e.g., 3D printing using 3D laser sintering, SLA printing, or the like). The methods may include 3D modelling the printhead prior to manufacturing the printhead. The methods may include designating a 3D model for the printhead. Optionally, the method may include modeling flow and mixing within the fluid mixer prior to manufacturing the printhead.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a schematic illustration of selected operations for preparing a mixture for microparticle generation.

FIG. 2A, FIG. 2B, and FIG. 2C provide schematic illustrations of printhead elements for component mixing and microparticle and/or nanoparticle generation.

FIG. 3 provides a schematic illustration showing a system for microparticle and or nanoparticle generation including a printhead for component mixing.

FIG. 4, FIG. 5, FIG. 6, FIG. 7, FIG. 8, FIG. 9, FIG. 10, FIG. 11, FIG. 12, and FIG. 13 provide schematic illustrations of printheads for component mixing and microparticle and/or nanoparticle generation.

FIG. 14 and FIG. 15 show flow simulations for MAGIC printheads.

FIG. 16, FIG. 17, FIG. 18, and FIG. 19 show dynamic light scattering (DLS) analysis results of particles generated from printheads according to examples described herein.

FIG. 20 provides data showing Circular Dichroism spectra obtained for fibroin particles fabricated according to examples described herein.

FIG. 21 provides data showing Fourier Transform-Infrared spectra for fibroin particles fabricated according to examples described herein.

FIG. 22, FIG. 23, FIG. 24, FIG. 25, FIG. 26, FIG. 27, and FIG. 28 show dynamic light scattering (DLS) analysis results of fibroin particles generated from printheads according to examples described herein.

FIG. 29 show scanning electron micrograph images of fibroin particles generated from printheads according to examples described herein.

FIG. 30, FIG. 31, and FIG. 32 provide schematic illustrations of printhead elements for component mixing and microparticle and/or nanoparticle generation.

### DETAILED DESCRIPTION

Extrusion-based 3D printing is often performed using a single ink or switching between two or more inks. The ability to create compositional variation in-situ by rapid mixing or biaxial or coaxial printing of two different inks inside a printhead with dynamically changing ratios can produce a broad palette of material compositions that are useful in making functionally graded materials for applications in, for example, soft robotics, tissue engineering, or integrated sensors. However, rapid combination of high viscosity bio-inks in low Reynolds number flows has been a challenge. In the present disclosure, multi-channeled and guided inner controlling (MAGIC) printheads are capable of coaxial, biaxial, and mixing dynamically controllable ratios of different bio-inks to print graded materials.

Bio-hydrogels have emerged as promising platforms for drug release systems due to inherent biocompatibility, controllable degradability, and tunable physical properties. Single contingent gel systems contain drug molecules that may lead to burst drug release. In order to develop a sustainable release hydrogel, a platform for drug delivery to targets organs (tissues) may be required. Herein, the coaxial hydrogel structures were developed that could simultaneously exert both affinity and diffusion control over the release of chemotherapeutic drugs. A range of coaxial hydrogel structures were developed through MAGIC 3D-printing techniques, with the purpose of building suitable therapeutic platforms for sustained and local release of drugs (e.g. anti-cancer drugs, anti-bacterial drugs, painkiller, etc.) to tissues/organs.

As one example, the printing systems described herein can be used to make a bi/tri cylinder that can load multiple drugs and release them at the same time. For example, a printed cylinder can permit one side to load lightweight and/or floating materials and the other side to load a drug, such that the resultant scaffold can float (e.g., in the stomach) with a particular orientation. Such aspects allow extension to new medical application areas such as floating tablets.

In examples, the present disclosure provides multi-channel controlled pneumatic printheads which contain a linker (e.g. Luer lock), a designed functional system (e.g., a microfluidic system, coaxial system, or a mixing system) and one or more outlets (e.g., nozzle part or output linker). Customized designs for the printheads can be created, for example, using computer aided design software and flow simulation software can be used to identify the extent and range of mixing within the printheads.

Air pressure controlled pneumatic printheads are capable of extruding a wide range of high and low viscosity materials. The connection of printheads with state-of-the-art 3D bioprinters allows for printing with a wide range of materials. In some examples, printers allow for delicate control of the temperature of the print bed and printhead, enabling a high level of printing quality, regardless of the bio-ink's viscosity.

Mixing is a process by which uniformity of concentration is achieved. Depending on the context, mixing may be in reference to the concentration of a particular component or set of components in a fluid. The design and implementation of mixers in microfluidics differs considerably from that on the macroscale. The small length scale leads to different physical phenomena being dominant at the microscale. First, inertial effects that typically result in turbulence and good mixing on the macroscale are weak in microfluidics, while methods of actuation based on electro kinetics, surface tension or other phenomena that are not relevant on the macroscale become feasible on the microscale. Secondly, many mechanical designs such as stirrers that can be easily implemented on the macroscale are very difficult to implement on the microscale. A useful mixer is therefore often one that is simple to fabricate and integrate with other microfluidic components.

The criteria used to measure mixing vary widely. One of the most common measures for mixing, known as the mixing variance coefficient (MVC), is based on the concentration distribution inside the channel or volume. For this purpose, the volume under consideration is divided into sub volumes, and the deviation of concentrations in each sub-volume from the average concentration in the volume is computed as follows:

$$M V C = 1 / N \sum {( {c}_{i} - {c}_{avg} )}^{2} \qquad ( 1 )$$

Here, N is the number of sub-domains, cᵢ is the concentration in the i^{th} sub-domain, and c_{avg} is the average concentration for the entire domain. MVC approaches zero when mixing is complete.

In some examples, methods for producing multi-channeled and guided inner controlling (MAGIC) printheads described herein can be performed by using a resin composition for stereolithography, for example, using liquid resin as a raw material. In some examples, printheads can be fabricated using SLA resin compositions using a variety of stereolithography methods including LCD (stereolithography liquid display method: Liquid Crystal Display), DLP (stereolithography projector (surface exposure) method: Digital Light Processing), and SLA (stereolithography laser method: Stereolithography Apparatus).

In some examples, printheads can be fabricated by using direct metal laser sintering (DMLS). DMLS is an industrial metal 3D printing process that builds fully functional metal prototypes and production parts in 7 days or less. A range of metals produce final parts that can be used for end-use applications. The DMLS machine begins sintering each layer-first the support structures to the base plate, then the part itself with a laser aimed onto a bed of metallic powder. After a cross-section layer of powder is micro-welded, the build platform shifts down, and a recoating blade moves across the platform to deposit the next layer of powder into an inert build chamber. The process is repeated layer by layer until the build is complete. When the build finishes, an initial brushing is manually administered to parts to remove most of the loose powder, followed by the appropriate heat-treat cycle while still fixtured in the support systems to relieve any stresses. Parts are removed from the platform and support structures are removed from the parts, then finished optionally using bead blasting and/or deburring. Final DMLS parts are near 100 percent dense.

Techniques described herein include those employing pneumatic, pressure assisted, extrusion-based 3D printing and emulsion/mixture evaporation for fabricating microparticles or nanoparticles, such as useful for particle-based drug delivery systems encapsulating an active pharmaceutical ingredient for the treatment of different diseases. The techniques provide for encapsulation of a variety of substances including proteins, plasmid DNA, lipophilic pharmaceutical compositions, hydrophilic pharmaceutical compositions, live cells, and/or cellular components into polymeric particles. A variety of biocompatible polymers can be used to formulate the particles, such as, but not limited to, poly(lactide-co-glycolide) (PLGA), polylactide (PLA), polycaprolactone (PCL), etc. Similarly, various non-polymeric excipients such as non-reducing sugars, such as trehalose, sucrose, or polyols (e.g., mannitol, sorbitol, xylitol), or amino acids (e.g., leucine) can be used as suitable carrier matrices to form the particles.

In some examples, described for purposes of illustrating the printing process in a general sense, an active pharmaceutical ingredient (if hydrophilic) can be dissolved in a poly(vinyl alcohol) (PVA) or other suitable polymeric aqueous solution including but not limited to, polyethylene glycol (PEG) or polyvinyl pyrrolidone (PVP), or lipidic solutions, optionally with a cosolvent to aid in dissolution in the case of hydrophobic active pharmaceutical ingredients. The active pharmaceutical ingredient dissolved in the aqueous PVA solution can be further added to a polymer dissolved in an organic solvent, such as chloroform, followed by mixing completely to form a primary mixture. The primary mixture can be further added to another PVA aqueous solution, such as with a higher PVA concentration, followed by mixing completely to generate a secondary mixture. The secondary mixture can be transferred to a pneumatic syringe with a fine gauge needle, then printed by a bioprinter employing an extrusion-based printing step. After the evaporation of the organic solvent, the resultant particles can optionally be washed (e.g., by ultracentrifugation) one or more times, and then collected.

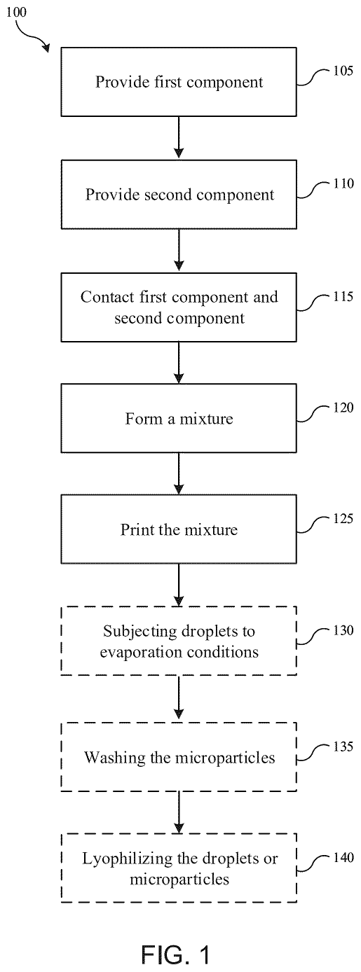

FIG. 1 provides a schematic overview of a process 100 of preparing mixtures according to various techniques described herein. Process 100 may include providing a first component at block 105 and providing a second component at block 110. The first component may include a polymer, a non-polymeric excipient, or an active pharmaceutical ingredient. For example, the active pharmaceutical ingredient may comprise a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or mega-nucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell. The polymer may be a biodegradable polymer, such as selected from the group consisting of poly(lactide-co-glycolide), polylactide (PLA), polyglycolide (PGA), polycaprolactone (PCL), Pluronic F127, sodium alginate, hyaluronic acid, chitosan, cyclodextrin, dextran, agarose, gelatin, albumin, collagen, fibroin lipids, a polyethylene glycol (PEG) derivative, a pharmaceutical grade polymer, poly(hydroxy butyrate), poly(β-malic acid), or poly(L-lysine). The non-polymeric excipient may be a hydrophilic substance, a hydrophobic substance, a non-reducing sugar, trehalose, sucrose, a polyol, mannitol, sorbitol, xylitol, an amino acid, leucine, or L-arginine. The second component may be a solvent.

At block 115, the first component and the second component are contacted. Contacting the first component and the second component may include providing the first component to an inlet of a fluid mixer in a printhead and providing the second component to the inlet of the fluid mixer in the printhead to form a mixture, such as at block 120. In some examples, the fluid mixer may be a coaxial arrangement of a first fluidic channel in fluid communication with a first linker and a second fluid channel in fluid communication with a second linker. The first component may be provided to the first linker, and the second component may be provided to the second linker, in order to feed the components to a mixing chamber or region (fluid mixer). Other configurations may be used, such as where two (or more) fluidic channels provide respective fluid pathways between two (or more) linkers and a mixing chamber (fluid mixer) in any suitable configuration. In some examples, the fluid mixer may be a stirrer. If present in the fluid mixer, a stirrer may be activated, such as by providing an electric field or a magnetic field. The fluid mixer may include a mixing architecture (e.g., flow pathway within the fluid mixer) characterized by an “S” shape or a helix shape. The mixture may be suitable for generating particles of active ingredients embedded in the materials using extrusion-based printing techniques described herein. Advantageously, the mixture can be prepared without the use of sonication or other ultrasonic mixing techniques that can result in raising the mixture temperature, allowing for temperature sensitive active ingredients to be incorporated into particles without being subjected to excessive temperatures.

At block 125, process 100 may include printing the mixture from the printhead through one or more outlets (e.g., printing nozzles). The printing may subject the mixture to shear forces that separate the mixture into a plurality of droplets including particles.

At block 130, process 100 optionally includes subjecting the droplets to evaporation conditions to evaporate the solvent from the droplets and leave the particles. At block 135, process 100 optionally includes washing the particles. At block 140, process 100 optionally includes lyophilizing the plurality of droplets or the particles.

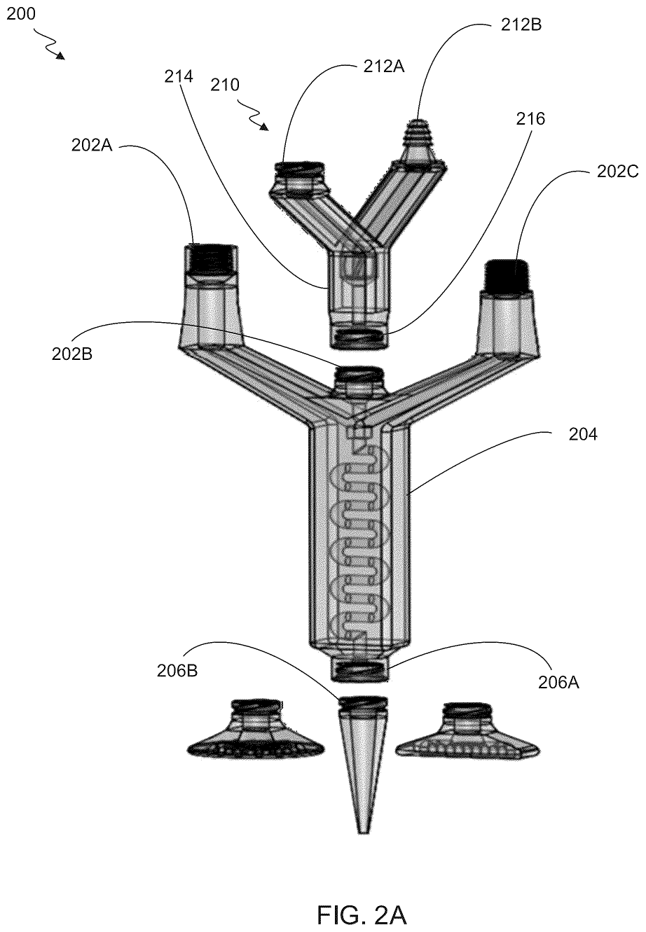

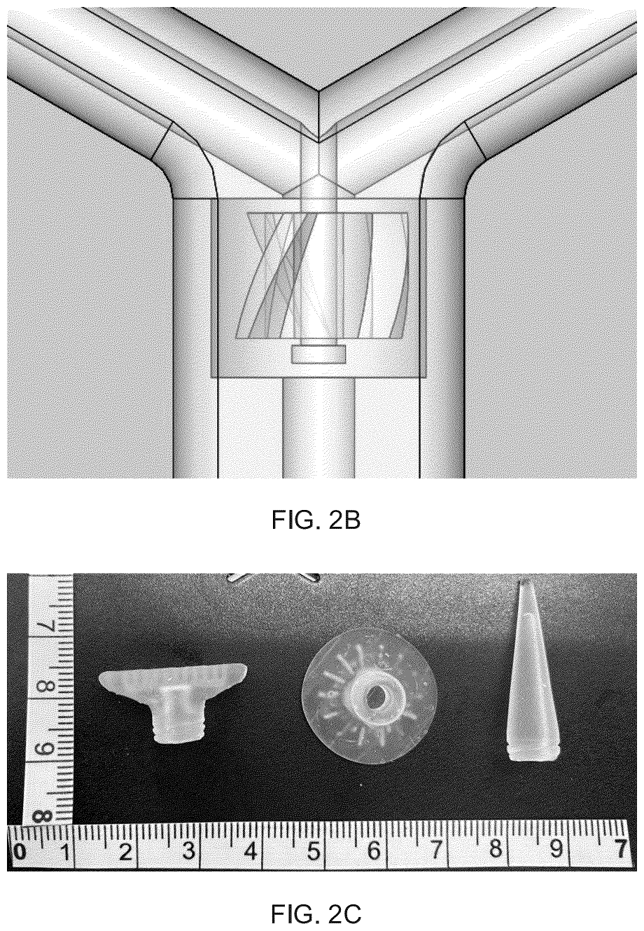

FIG. 2A provides a schematic depiction of a printhead 200 for generating particles. The printhead 200 may include a plurality of linkers 202A, 202B, 202C defining inlets of the printhead 200. The printhead 200 may include a fluid mixer 204 in fluid communication with the plurality of linkers 202A, 202B, 202C for mixing components from the inlets into a mixture. The fluid mixer 204 may be in fluid communication with the plurality of linkers 202A, 202B, 202C via a plurality of primary fluid lumens.

The printhead 200 may include one or more outlets in fluid communication with the fluid mixer 204. The one or more outlets may be in fluid communication with the fluid mixer 204 via a one or more secondary fluid lumens. As depicted in FIG. 2A, the outlet of component 200 includes an outlet linker 206A and a nozzle 206B. Although outlet linker 206A and a nozzle 206B are shown as two separate components, the outlet linker 206A and a nozzle 206B may be a unitary structure with fluid mixer 204 such that no outlet linker 206A is present and the nozzle 206B is joined to the fluid mixer 204. FIG. 2A also shows other nozzle configurations on opposite sides of nozzle 206B, which may be substituted for nozzle 206B. The other nozzle configurations may include a plurality of nozzle ports, while nozzle 206B may include a single nozzle port.

Printhead 200 is also shown including sub-printhead 210, which includes linkers 212A and 212B, fluid mixer 214 and outlet 216. Outlet 216 is depicted as an outlet linker, which may be coupled to the central linker 202B, for example. Such sub-printhead 210 is depicted to illustrate a configuration where multiple printheads are coupled to one another, so as to provide additional opportunities and configurations for mixing additional components before they are introduced into a fluid mixer.

One or more of the plurality of linkers 202A, 202B, 202C, 212A, 212B may include a Luer lock, a Luer slip, a slip tip, or other couplers for establishing sealable fluid communication. As shown in FIG. 2A, the plurality of linkers 202A, 202B, 202C, 212A, 212B may be various types of couplings or connectors. However, it is also contemplated that each of the plurality of linkers 202A, 202B, 202C, 212A, 212B may be the same type of coupling or connector. The linkers 202A, 202B, 202C, 212A, 212B may be characterized by a diameter of less than or about 5.0 mm, such as less than or about 4.5 mm, less than or about 4.0 mm, less than or about 3.5 mm, less than or about 3.0 mm, less than or about 2.5 mm, less than or about 2.0 mm, less than or about 1.5 mm, less than or about 1.4 mm, less than or about 1.3 mm, less than or about 1.2, mm, less than or about 1.1 mm, less than or about 1.0 mm, less than or about 0.9 mm, less than or about 0.8 mm, less than or about 0.7 mm, less than or about 0.6 mm, less than or about 0.5 mm, or less.

The fluid mixer 204 may be a coaxial arrangement of a first fluidic channel in fluid communication with a first linker and a second fluid channel in fluid communication with a second linker. In some examples, the fluid mixer may include a stirrer as shown in FIG. 2B. The mixer may be activated using any means, including an electric field or a magnetic field for activating the stirrer. While the stirrer is shown at an upstream location of the fluid mixer, it is contemplated the stirrer may be positioned at any location along the fluid mixer. Further, it is contemplated that a plurality of fluid mixers, such as stirrers, may be used to form the mixture. The fluid mixer may include a mixing architecture characterized by a “Y” shape, an “S” shape, a helix shape, or any other desirable shape to establish a desired amount of mixing of components introduced from various linkers.

The one or more outlets or nozzles 206A, 206B, 216 of the printhead 200 or sub-printhead 216 may be characterized by a diameter of less than or about 5.0 mm, such as less than or about 4.5 mm, less than or about 4.0 mm, less than or about 3.5 mm, less than or about 3.0 mm, less than or about 2.5 mm, less than or about 2.0 mm, less than or about 1.5 mm, less than or about 1.4 mm, less than or about 1.3 mm, less than or about 1.2, mm, less than or about 1.1 mm, less than or about 1.0 mm, less than or about 0.9 mm, less than or about 0.8 mm, less than or about 0.7 mm, less than or about 0.6 mm, less than or about 0.5 mm, or less. The one or more outlets may include one or more extrusion-based printing nozzles, such as nozzle 206B, for generating a plurality of droplets of the mixture including particles having diameters of from 10 nm to 1100 μm. FIG. 2C shows photographs of various nozzles including nozzle linkers for joining to an output linker of a printhead. Without limitation, the outlet of the printhead 200 may include one or more outlets, such as in the form of one or more nozzle ports.

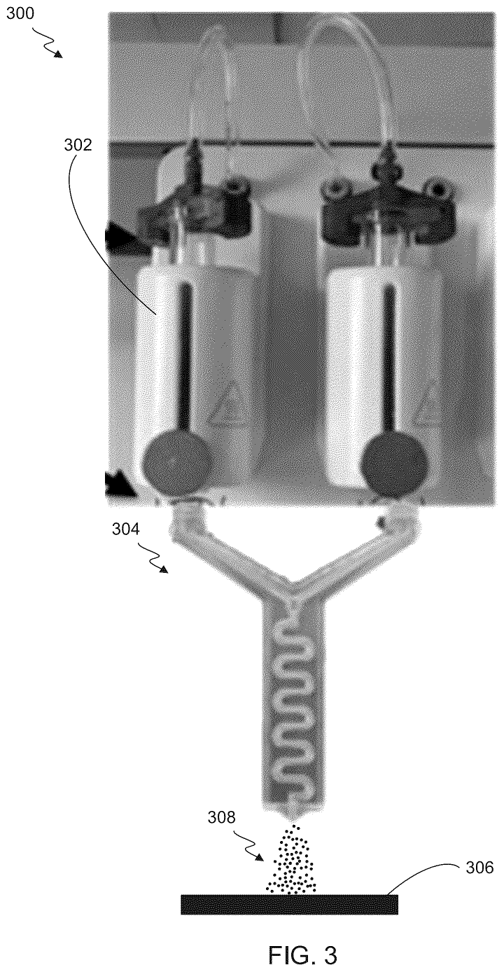

As shown in FIG. 3, a system 300 for generating particles is schematically depicted. The system may include a plurality of supply containers 302 for preparing or storing a respective component, such as comprising a polymer or a non-polymeric excipient, a solvent, or an active pharmaceutical ingredient. The system 300 may also include a printhead 304, which may include any of the features or characteristics previously discussed with regard to printhead 200 or other printheads described herein. The system 300 may include a collection surface 306 for receiving a plurality of droplets 308 of the mixture from the one or more outlets of the printhead, such as droplets 308 that contain particles. The collection surface 306 may be, for example, a sterile vial.

The collection surface 306 of system 300 may be cooled to a temperature of from about −200° C. to about −75° C. during operation. For example, a cooling or refrigeration system coupled to the collection surface 306 may cool the collection surface to a temperature of from about −200° C. to about −75° C. One or more temperature sensors or temperature controllers may monitor or control a temperature of the collection surface 306. In examples, the collection surface 306 may be a moving or translating collection surface.

System 300 may optionally include one or more pressure sensors or pressure controllers, temperature sensors or temperature controller, position sensors, translation stages, or other sensors or controllers. In some examples, the temperature may be controlled over a wide range, such as from about −120° C. to about 250° C., the printhead 304 may be compatible with use of a wide range of FDA approved thermoplastics or for establishing low temperature, useful to maintain viability of cells or other biomaterial in the mixture to be printed using the printhead. As an example, for use with heated mixtures, the printhead can be used to extrude thermoplastic materials, such as for making pharmaceutical tablets with crystal drugs (e.g., biopharmaceutics classification system class 3). As another example, for use with cooled mixtures, the printhead is compatible with temperature sensitive biomaterials. As examples, the system 300 may include a Peltier cooler, a heat exchange jacket, or other heat exchanger in contact with the printhead 304 or fluid mixer region, such as adjacent to or surrounding the S-shaped flow path of the fluid mixer depicted, to add or remove heat and/or control temperature of the mixture within the printhead 304. In some examples, water from a bath can be circulated in a heat exchange apparatus which surrounds the fluid mixer or fabricated as flow paths within the periphery of the fluid mixer.

The present disclosure also encompasses methods of creating, designing, forming, and/or fabricating printheads, such as printhead 200 previously described. The methods may include manufacturing a printhead, the printhead including a plurality of linkers defining inlets of the printhead, a fluid mixer in fluid communication with the plurality of linkers for mixing components from the inlets into a mixture, and one or more outlets in fluid communication with the fluid mixer. Manufacturing the printhead may include additive manufacturing such as 3D laser sintering, SLA printing, or the like. The methods may include 3D modeling the printhead prior to manufacturing the printhead. The methods may include designating a 3D model for the printhead. The methods may also include studying properties of the printhead prior to manufacturing, such as modeling flow and mixing within the fluid mixer, and droplet formation via one or more nozzles or nozzle ports prior to manufacturing the printhead. The processes of modeling and designating a 3D model for the printhead and modeling flow, mixing, and printing may be performed iteratively, such as to optimize the printhead for printing droplets or particles of particular mixtures or mixtures of particular properties.

In some examples, the printheads, methods, and systems described herein are useful for preparing microparticles and/or nanoparticles, such as using sprayed multi adsorbed-droplet reposing technology. Example techniques and particle components and compositions are described in U.S. Provisional Application No. 63/219,258, filed on Jul. 7, 2021, and PCT International Application No. PCT/US2022/036336, filed on Jul. 7, 2022, which are hereby incorporated by reference in their entireties.

The invention may be further understood by the following non-limiting examples.

### Example 1—Magic Printheads and Example Particles

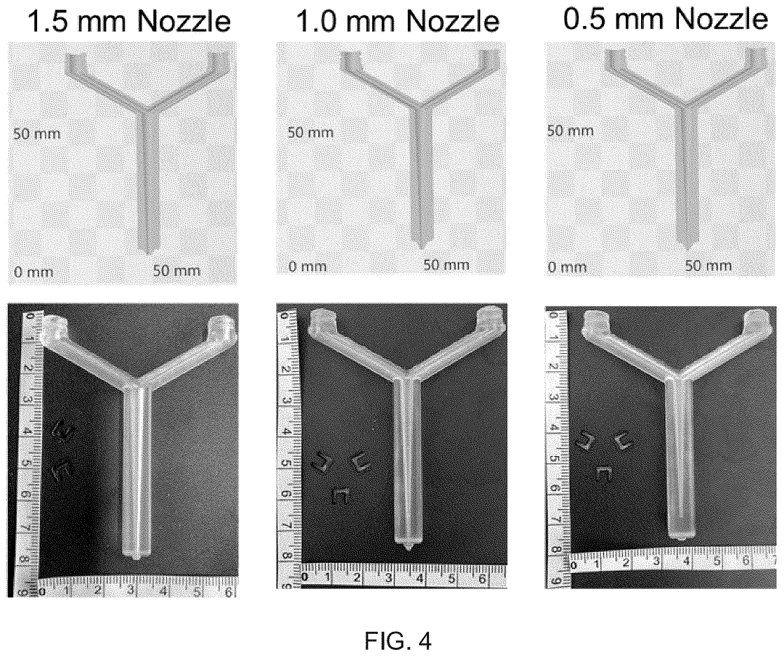

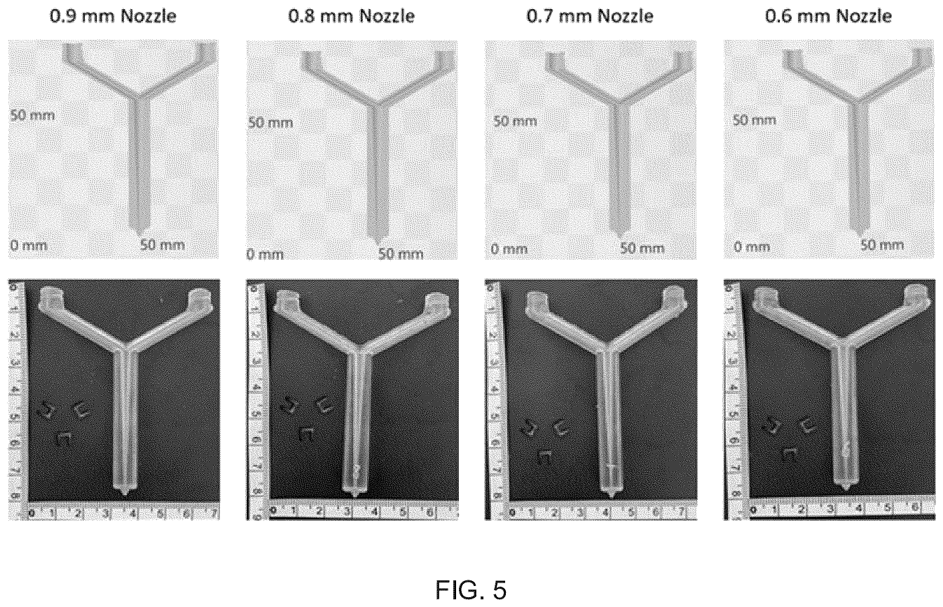

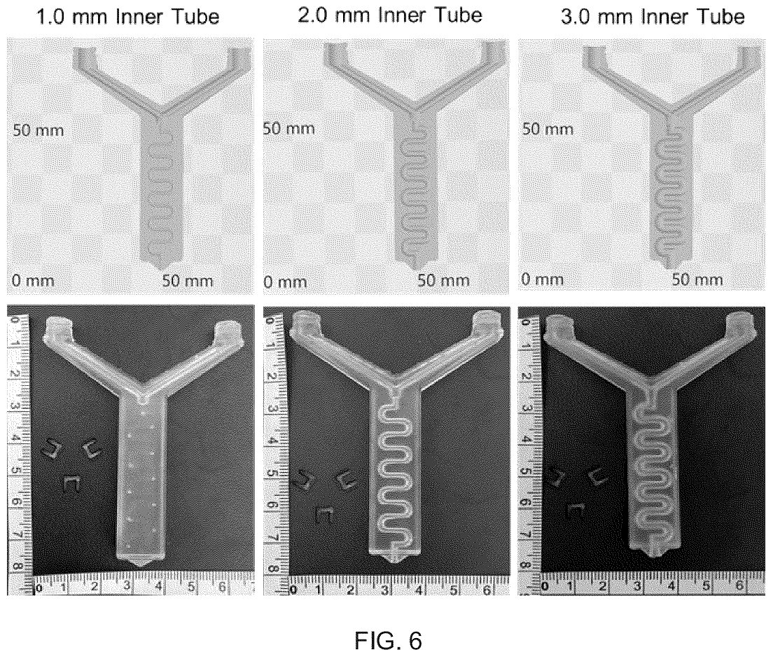

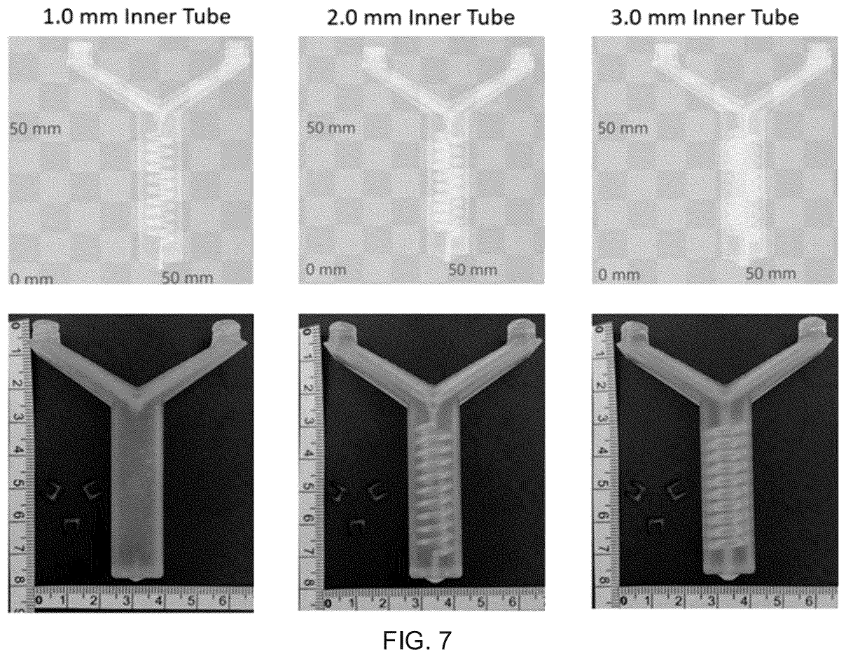

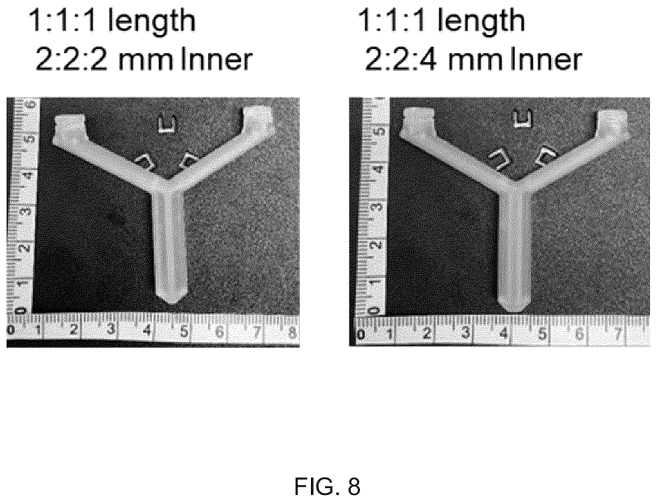

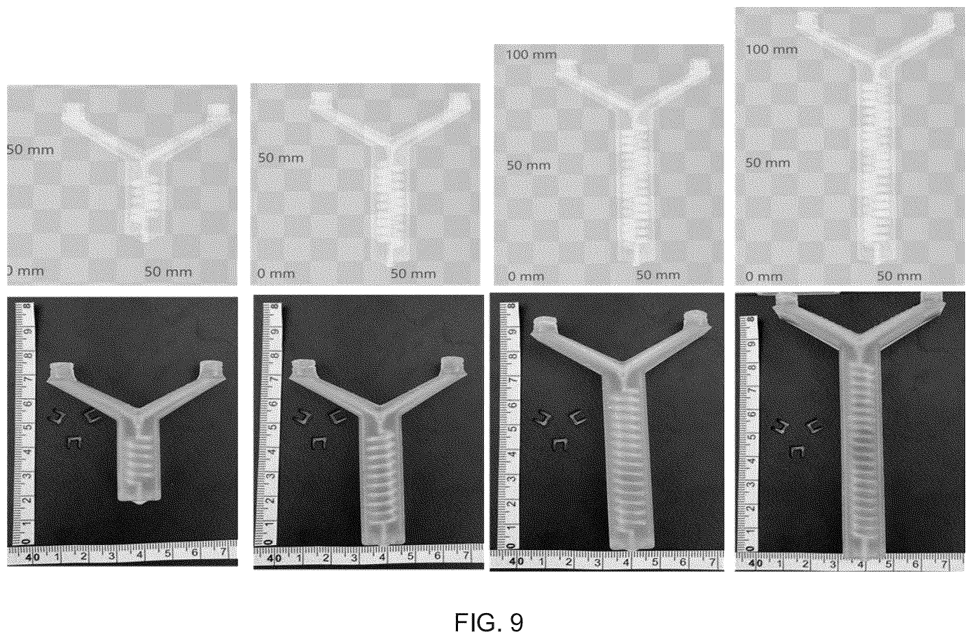

FIG. 4, FIG. 5, FIG. 6, FIG. 7, FIG. 8, FIG. 9, FIG. 10, FIG. 11, FIG. 12, and FIG. 13 provide numerous views of printheads, including various fluid mixer types and architectures, as well as various dimensions. The top rows in FIGS. 4-7 show depictions of 3D models of the printheads, while the bottom rows in FIGS. 4-7 show photographs of fabricated printheads, created using SLA printing techniques using a standard clear SLA resin. These different printheads were used to establish the manufacturing limits according to the particular SLA printing system used, to identify the smallest flow path and/or nozzle sizes.

FIG. 8 shows fabricated printheads with various nozzle sizes. The flow rate may be controlled by the input pressure. The flow rate is one parameter that is related to the size of the final particles. FIG. 9 shows 3D helix shaped MAGIC printheads with various length (e.g., the number of coils). In FIG. 9, the number of coils are as follows: A) 5 coils, (B) 10 coils, (C) 15 coils, and (d) 20 coils. All inner tubes in FIG. 9 have a fixed 2 mm diameter. FIG. 10 shows fabricated coaxial printheads with various inner length. In FIG. 10, the inner lengths are as follows: A) 10 mm, (B) 15 mm, (C) 20 mm, and (d) 30 mm. All outer lengths in FIG. 10 are 30 mm. FIG. 11 shows printheads with multiple inputs (e.g., three or more). For example, the printhead may include three inputs or four inputs, or any number of inputs. In FIG. 11, the diameter for the input is 1 mm.

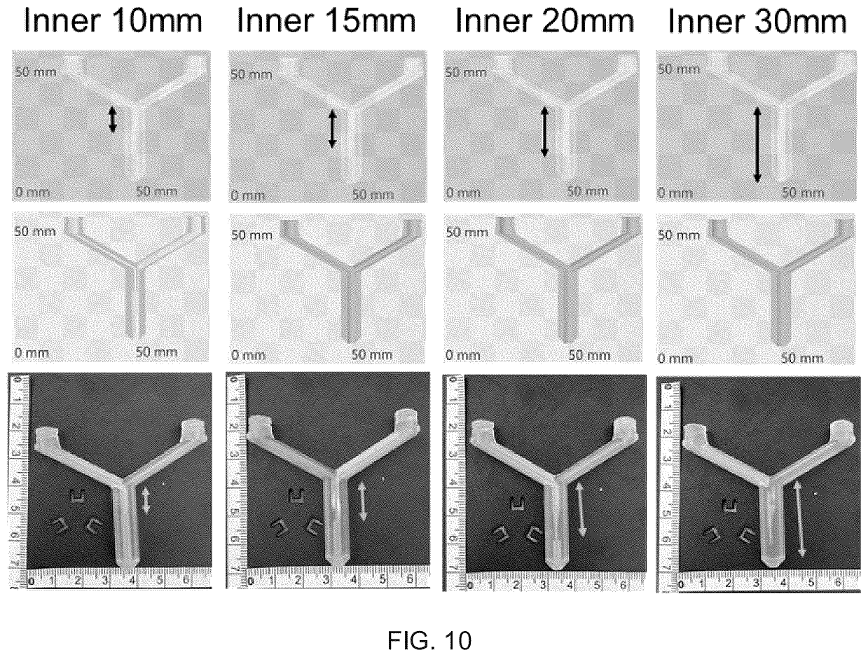

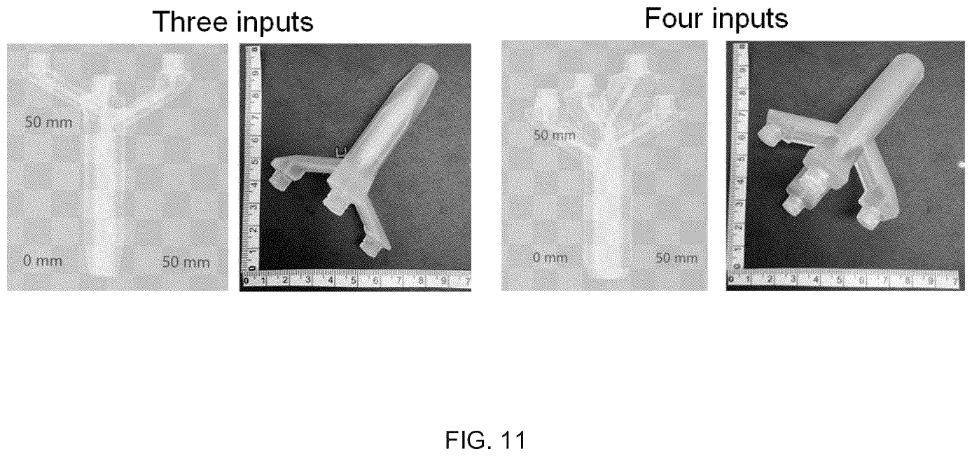

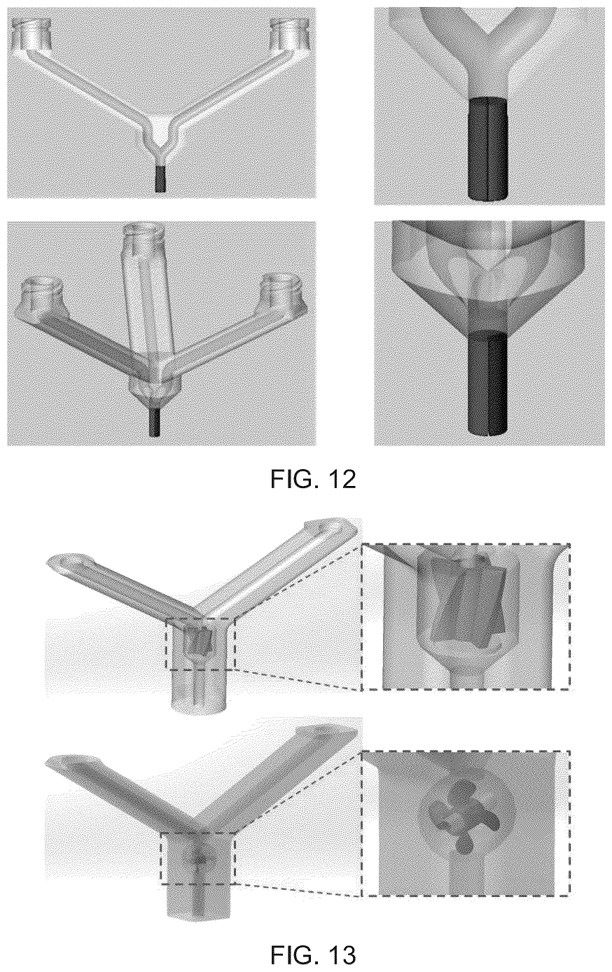

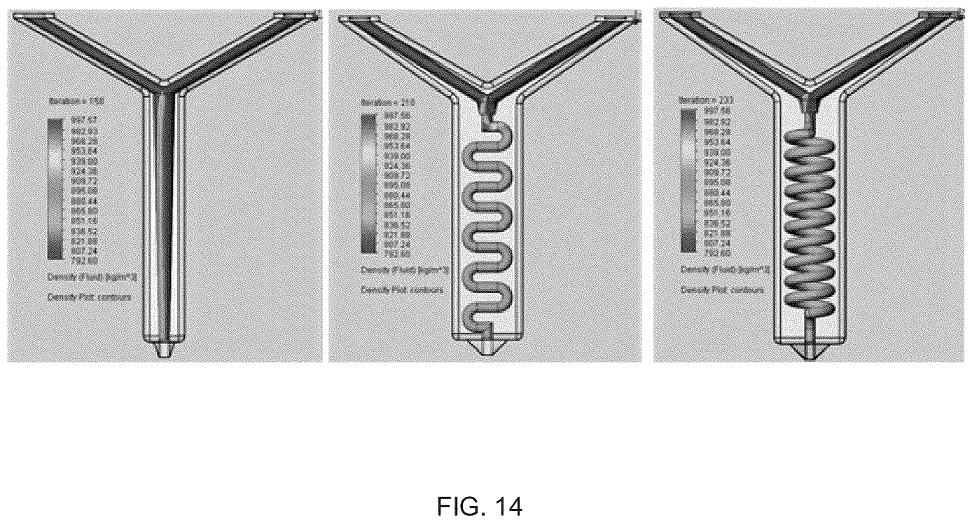

3D flow simulations were performed for each of the printheads depicted in FIGS. 4-11 to evaluate flow conditions (e.g., flow rate, flow direction, pressure, shear force, density, viscosity, etc.) in the printhead system and the extent of mixing. FIG. 12 provides a schematic of the mixing in a printhead with two or three inputs. FIG. 13 provides example stirrers that may be used in the printhead to encourage mixing. FIG. 14 provides data showing the extent of mixing determined by the flow simulations for several printhead designs, including a colinear fluid mixer, a fluid mixer characterized by an “S” shape, and a fluid mixer characterized by a 3D helix. The flow simulation mimics the changing of the flow density via the microfluidic system.

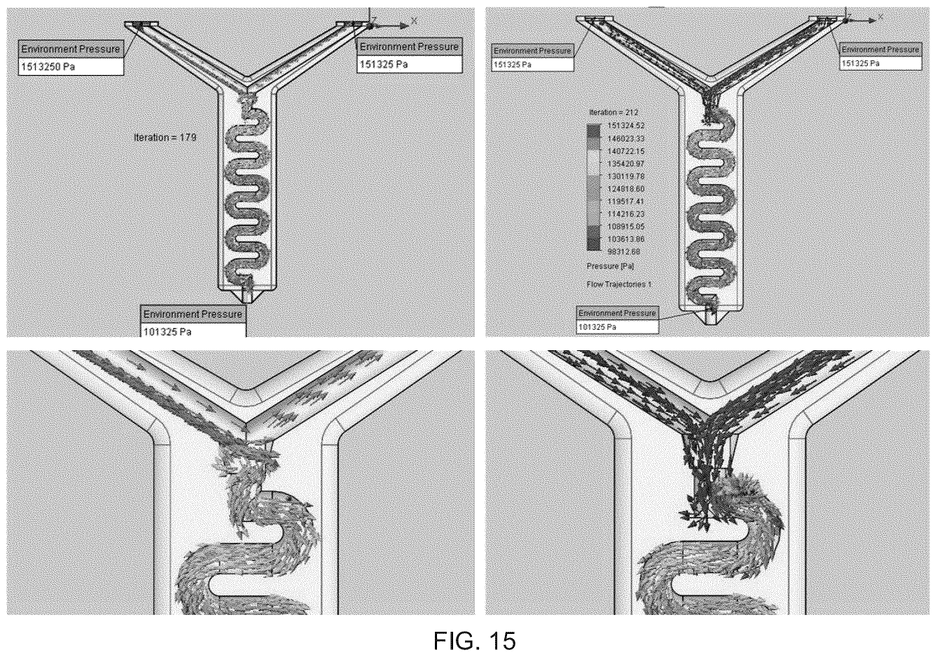

FIG. 15 shows additional details from the flow simulation for printheads having a fluid mixer characterized by an “S” shape for different pressure conditions at the fluid inlets, identifying flow trajectories and pressures within the different regions of the printhead, showing how the flow simulation mimics the flow direction under different input pressure.

Particles in liquid suspension were evaluated at 25° C. using a Zeta sizer Nano ZS (Malvern Instruments). A 633 nm, He—Ne laser was used as the light source while an avalanche photodiode (APD) served as the detector. Particle sizes were measured using dynamic light scattering (DLS) method where the scattered light was collected at 173°. The Z-Ave value was reported as the mean diameter of nanoparticles where the cumulant method was adopted for data analysis.

Protein microparticles are prepared according to the following process. A first component includes a regenerated Bombyx mori silk fibroin aqueous solution. A second component includes an organic solvent, including, but not limited to, one or more of ethanol, methanol, acetone, isopropanol, etc. Then, the first and second components are transferred to two pneumatic syringes and connected to a printhead by the top linkers, separately. Both components print by a bioprinter employing an extrusion-based printing step. For some examples, 0.1 mg/ml regenerated silk fibroin solution was used as the first component.

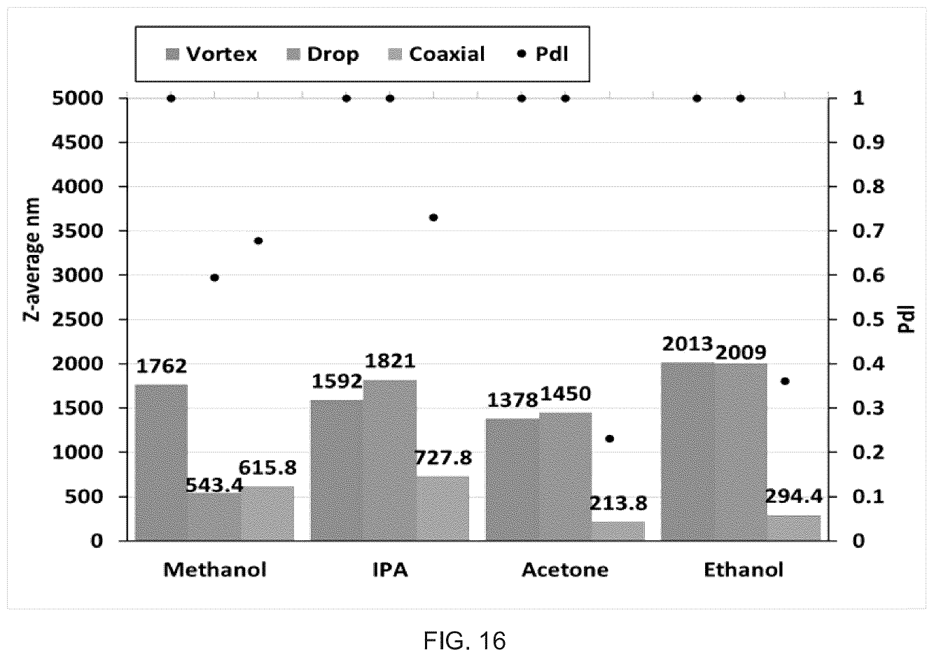

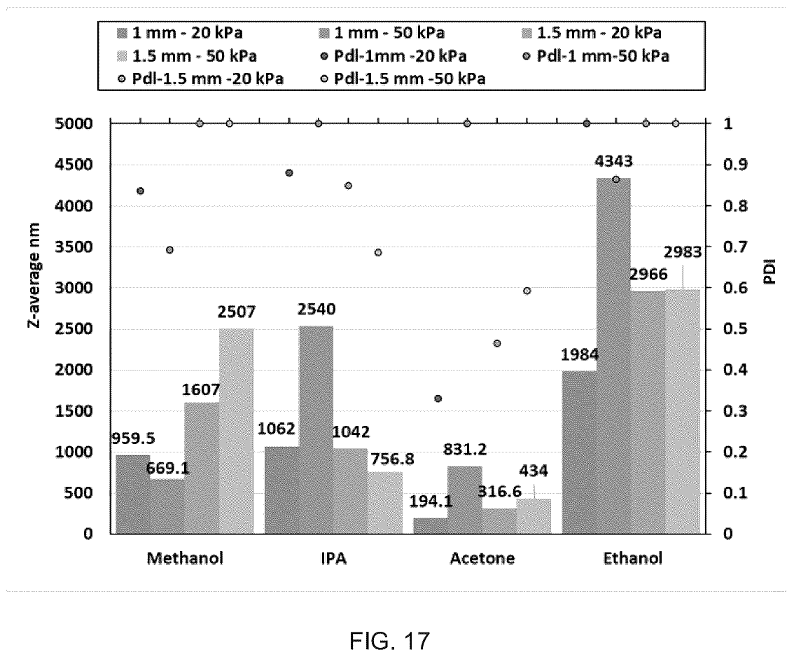

FIG. 16 shows differential light scattering (DLS) analysis of the particles made from vertexing, dropping, and coaxial printing. These three methods of making particles were used for comparing to methods of printing using the printheads described herein.

FIG. 17 shows DLS analysis of the particles made using a “Y” shaped printhead described herein. Printheads with nozzle size 1.5 mm and 1.0 mm diameter were used. 20 kPa and 50 kPa extraction pressures were tested separately with both “Y” shaped printheads. Silk fibroin and various organic solvents (methanol, isopropanol, acetone, and ethanol) were selected and analysis of the particles using various different nozzle sizes, pressures, and solvents and are displayed in FIG. 17.

FIG. 18 shows DLS analysis of the particles made from 3D-helix shaped printhead fluid mixers described herein and compared with those with “Y” shaped printheads. Printheads with nozzle sizes 1.5 mm and 0.8 mm diameter were used for the “Y” shaped printheads, with 3 mm diameter nozzle size used for the 3D helical printhead. PLGA in acetone and PVA in deionized water were used for the components.

FIG. 19 show DLS analysis results of particles generated from printheads according to examples described herein.

### Example 2—Silk Particles

Protein-based particles may be useful as drug delivery vehicles (DDVs) due to their biocompatibility. In some examples, fibroin-protein-based DDVs have gained attention due to their biodegradability, excellent mechanical properties, processing flexibility, and ‘green’ regeneration properties. However, no convincing report has investigated the fabrication mechanism of silk fibroin particles (SFPs) as a vehicle for enzyme/drug delivery system. This Example describes use of the Multi-Channeled and Guided Inner Controlling (MAGIC) techniques described herein to synthesize the SFPs. Specifically, this Example describes a quality-by-design (QbD) study to identify correlations between the particle size and organic solvents (ethanol, methanol, isopropanol, acetone, etc.), design of the MAGIC system (simple ‘Y’ shape, 2D zigzag shape, 3D spiral shape, etc.), or printing parameters (pressure or nozzle size), respectively. The size of the generated particles was analyzed by dynamic light scattering (DLS), and scanning electron microscopy (SEM). The physico-chemical characterization of the SFPs was analyzed by circular dichroism (CD) Spectroscopy and Fourier Transform-Infrared (FTIR) Spectroscopy. In addition, a fluid simulation model (Solidworks) has been used to study the designed system's mixing process in a 1 mm, 2 mm, or 3 mm-μm-wide channel integrating a MAGIC printing system by a standard derivation of color index at individual pixels. The concentration distribution was obtained by successfully solving the Navier-Stokes equation and the diffusion-convection equation in the steady-state form. Because of the large range of Reynolds numbers studied (100<Re<600), the diffusion-convection simulations are carried out with high diffusion coefficients. Accordingly, mixing indexes in the channel flow are compared at different cross sections and the relationship between the mixing quality and size of the SFPs are calculated. The results illustrated the effects of both pressure and channel geometry on hydrodynamics and mixing efficiency. Finally, applying the MAGIC system for making particles as active pharmaceutical ingredients (APIs) carriers for drug delivery systems, proximal tubule cells (PTC) protection surfactants, and tissue engineering are evaluated.

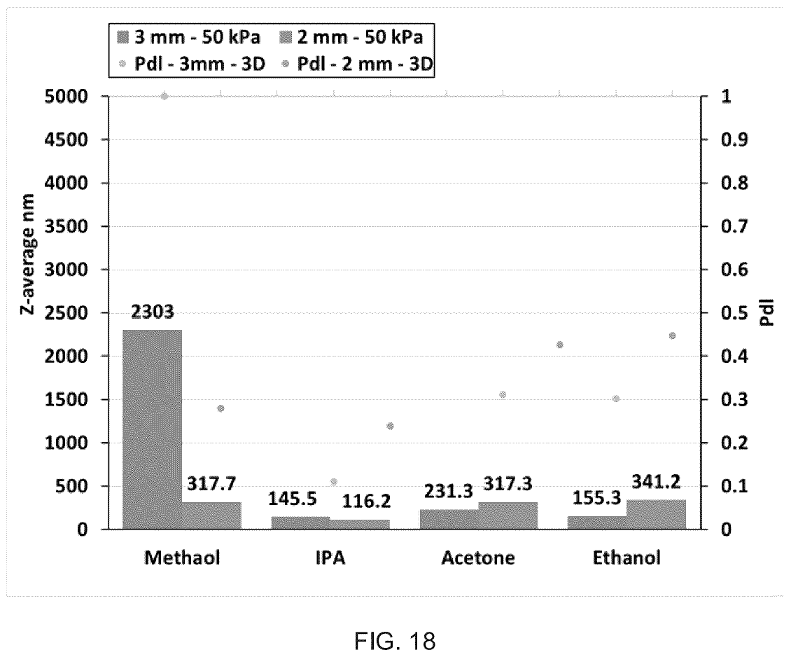

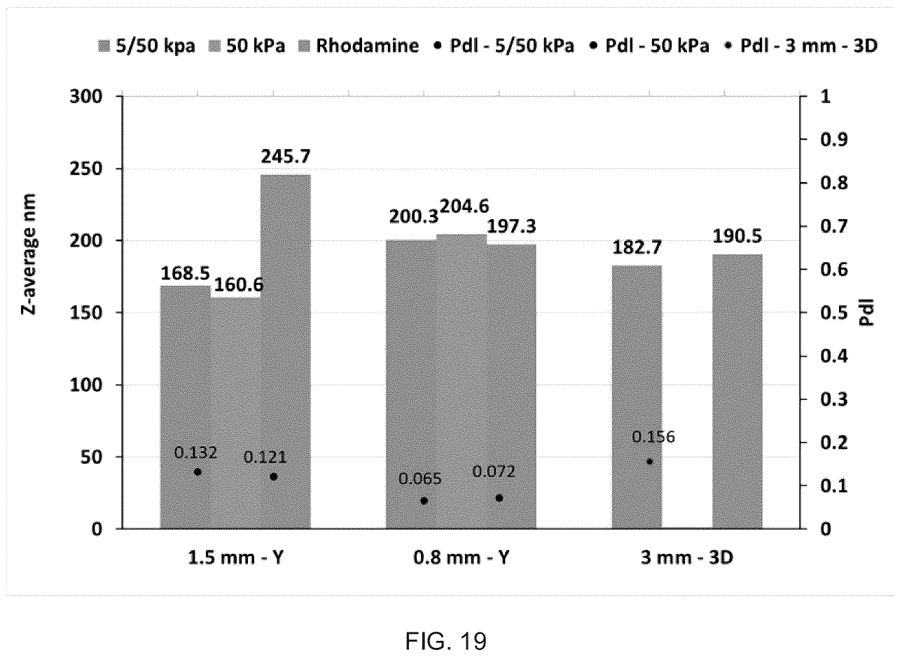

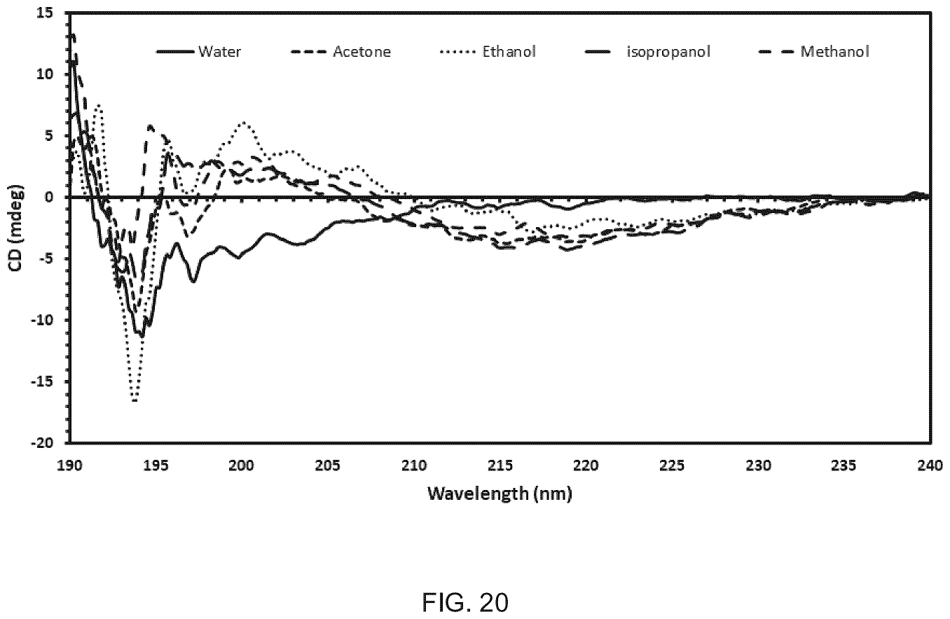

Circular Dichroism (CD) Spectroscopy. The fabricated fibroin particles were suspended in acetone, ethanol, methanol, or isopropanol solvent with a final concentration of 0.1 mg/mL. Circular Dichroism (CD) spectroscopy was used to investigate the secondary structure of the encapsulated trypsin. A JASCO-810 Spectrometer (Japan) was equipped with RTE bath/circulator (NESLAB RTE-111) and purged with N₂ gas at a flow-rate of 3-5 mL/min. The CD spectrometer was used for the scanning, which was carried out for 190-260 nm wavelengths with a resolution of 0.2 nm at 25° C. and accumulation of six scans. The scan speed was 100 nm/min and the response time was 0.25 s. Six measurements were carried out to obtain each spectrum. Samples of the 0.05 mg/mL and 0.01 mg/mL solutions were stored in 0.1-cm and 1-cm path length cells, respectively. The mean residue ellipticity values [Φ] were expressed in degree·cm²·dmol⁻¹ and were calculated using the equation:

$$[ Θ ] = Θobs * 78 / ( 10 dc )$$

where Φobs is the observed ellipticity value in degrees, the value 78 is the mean residue molecular mass taken from sequence data for silkworm protein, d is the optical path length in centimeters, and c is the protein concentration in grams per milliliter. FIG. 20 provides a plot showing Circular Dichroism spectra obtained for the fibroin particles fabricated using the MAGIC system.

Fourier Transform-Infrared (FTIR) Spectroscopic Analysis. A modular Nicolet™ iSTM 50 Fourier Transform Infrared (FTIR) spectroscopy (ThermoFisher Scientific, Waltham, Massachusetts, USA) was used to investigate the chemical structure of silk fibroin and MAGIC printed SFPs. To this aim, a liquid sample (100 μl of each sample) of the fibroin solution, SFPs produced by MAGIC with acetone, ethanol, methanol, or isopropanol were analyzed for percentage transmittance from 4000 to 400 cm⁻¹, at a resolution of 4 cm⁻¹ and 64 scans per run. The absorbance mode was used. WINFIRST series software (Version 9.0 ThermoFisher Scientific, Waltham, MA, USA) was used to measure and analyze the spectra.

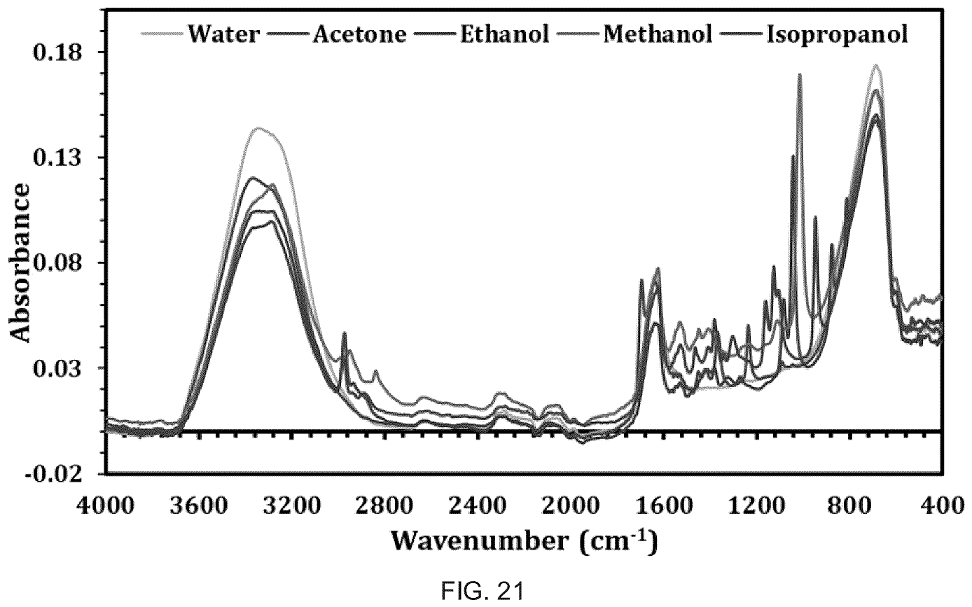

The infrared (IR) spectral region between 1750 cm⁻¹ and 1450 cm⁻¹ was classified as absorption by the peptide backbones of amide I (1700-1600 cm⁻¹) and amide II (1600-1500 cm⁻¹), which were mostly used for the analysis of different secondary structures of RSF. The FTIR spectra are shown in FIG. 21, where the peaks at 1661-1663 cm⁻¹, 1575-1777 cm⁻¹, and 1525-1522 cm⁻¹ are characteristic of silk II secondary structure, whereas the absorptions at 1672-1669 cm⁻¹ and 1531-1529 cm⁻¹ are indicative of silk I conformation. Upon printing using methanol, the peaks at 1670 cm⁻¹ and 1530 cm⁻¹ (silk I) decreased, whereas the peaks at 1662 cm⁻¹ and 1524 cm⁻¹ (silk II) increased, indicating that silk films with different amounts of crystal structures can be achieved by mixing with an organic solvent.

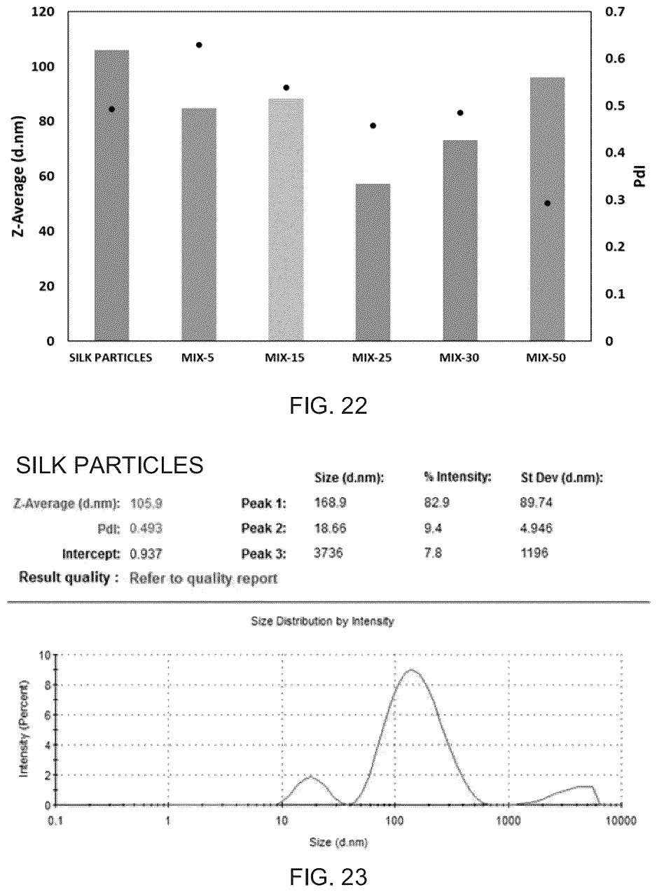

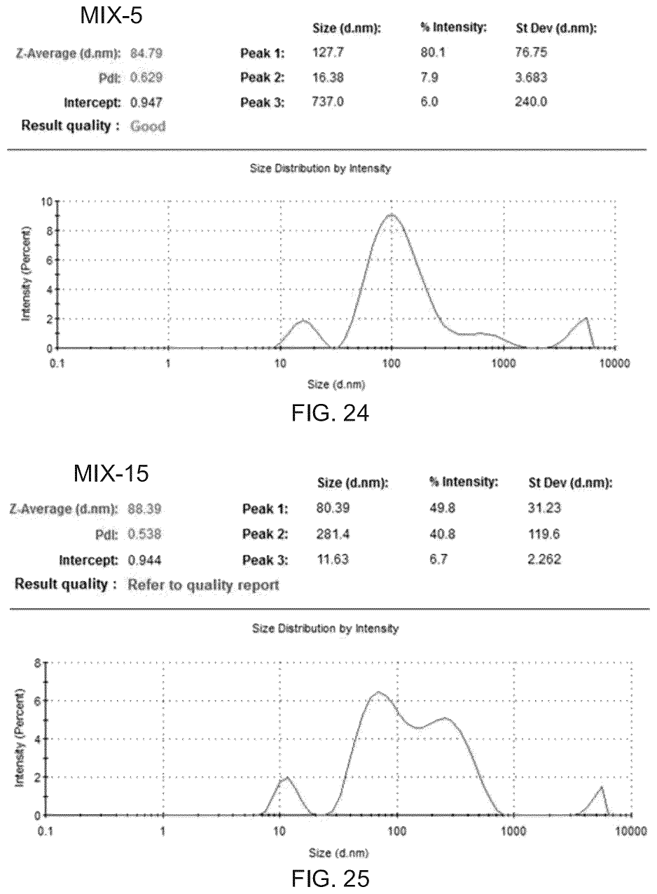

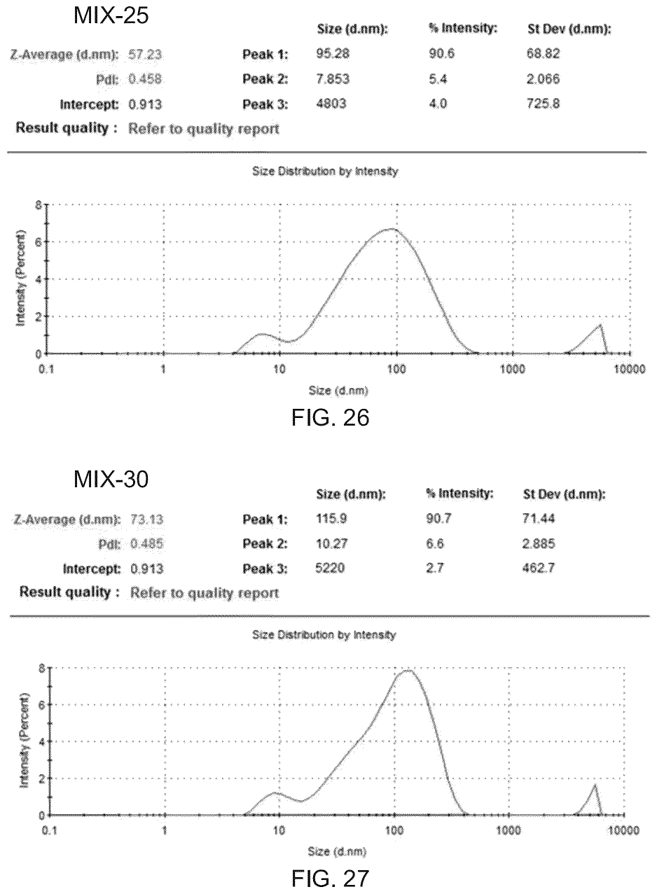

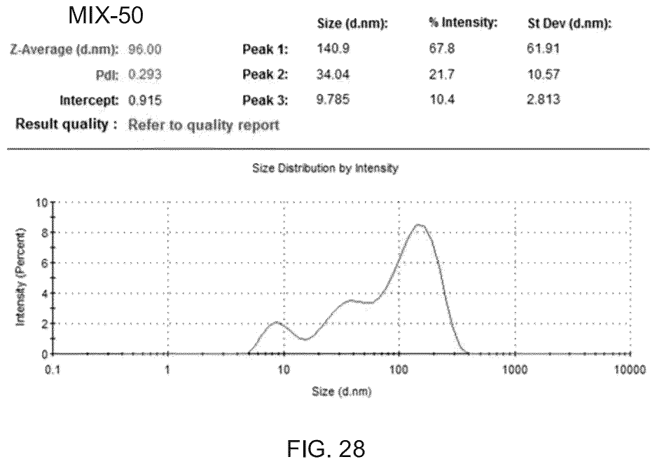

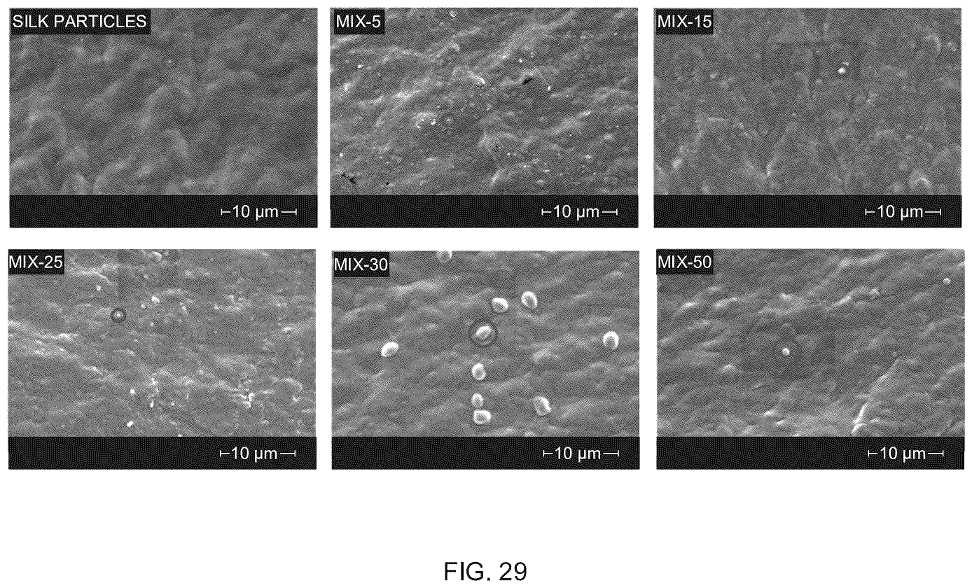

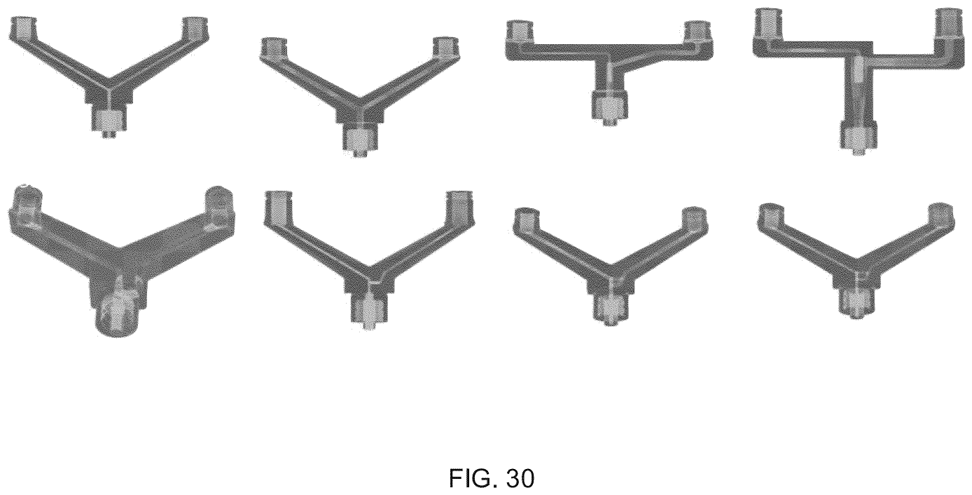

Silk particles cargos. A variety of particles comprising silk fibroin were prepared using a MAGIC system employing ethanol as a solvent, with varying amounts of another component, dimethylsulfoxide (DMSO). The various formulations tested are listed in Table 1. The particles were analyzed using dynamic light scattering (DLS), and scanning electron microscopy (SEM). Results from the dynamic light scattering measurements are shown in FIG. 22, FIG. 23, FIG. 24, FIG. 25, FIG. 26, FIG. 27, and FIG. 28. FIG. 29 shows scanning electron microscopy images of the particles, with an individual particle circled in the image as an example.

TABLE 1
Silk fibroin particle formulations

| | mg/ml fibroin | v/v % DMSO | |
|---|---|---|---|
| Sample ID | stock | in EtOH | F:D |
| SILK PARTICLES | 10 (3 ml) | 0 (9 ml) | 1:3 |
| MIX-5 | 10 (3 ml) | 10 (9 ml) | 1:3 |
| MIX-15 | 10 (3 ml) | 15 (9 ml) | 1:3 |
| MIX-25 | 10 (3 ml) | 25 (9 ml) | 1:3 |
| MIX-30 | 10 (3 ml) | 30 (9 ml) | 1:3 |
| MIX-50 | 10 (3 ml) | 50 (9 ml) | 1:3 |

### Example 3—Lipid/Liposome Magic System

Encapsulating mRNA in lipid nanoparticles. Encapsulating mRNA in lipid nanoparticles is a valuable technique used in mRNA-based vaccines and therapies. Lipid nanoparticles protect the fragile mRNA molecules and facilitate their delivery into cells, where they can be translated into proteins or used for gene therapy. Encapsulation of mRNA in lipid nanoparticles may include one or more of the following:
a. Selection of Lipids: Suitable lipids may be chosen for nanoparticle formation. Example lipids may include a mixture of cationic lipids (positively charged) and helper lipids. Cationic lipids help to bind and condense the negatively charged mRNA, while helper lipids stabilize the nanoparticle structure and improve delivery efficiency.
b. Preparation of Lipid Solution: The selected lipids may be dissolved in an organic solvent, such as ethanol or chloroform, to create a lipid solution.
c. Formation of Lipid-MRNA Complex: The mRNA molecule may be combined with the lipid solution. The positively charged cationic lipids will electrostatically interact with the negatively charged mRNA, forming a lipid-mRNA complex.
d. Nanoparticle Formation: An aqueous solution (buffer) may be added to the lipid-mRNA complex and subject the mixture to controlled agitation or sonication. This process can lead to the spontaneous formation of lipid nanoparticles, with the mRNA encapsulated within their core. This may include using a MAGIC system as a new nanoparticle formation system for lipid-mRNA system.
e. Purification: The lipid nanoparticles may be purified to remove any excess lipids, free mRNA, or other impurities. Example purification methods include ultrafiltration or dialysis.
f. Characterization: The lipid nanoparticles may be analyzed to ensure their size, stability, and encapsulation efficiency. Techniques such as dynamic light scattering and transmission electron microscopy can be used for characterization.
g. Storage: The encapsulated mRNA in lipid nanoparticles may be stored at appropriate conditions (e.g., low or subzero temperatures) to maintain stability until they are ready for use.

It will be appreciated that the specific process of encapsulating mRNA in lipid nanoparticles can vary depending on the intended application and the specific lipids used.

Encapsulating mRNA in liposome particles. Encapsulating mRNA in liposomes is a technique used for mRNA delivery in various applications, including gene therapy and vaccine development. Liposomes are lipid-based vesicles that can encapsulate the mRNA and protect it from degradation, facilitating its delivery into cells. Encapsulation of mRNA in liposomes nanoparticles may include one or more of the following:
a. Selection of Lipids: Appropriate lipids may be chosen for liposome formation. Lipids with varying properties, such as cationic lipids or neutral lipids, can be used to tailor the characteristics of the liposomes for specific applications.
b. Preparation of Lipid Solution: The selected lipids may be dissolved in an organic solvent, such as chloroform or ethanol, to create a lipid solution.
c. Formation of Liposomes: The lipid solution may be combined with the mRNA solution. The lipids may spontaneously self-assemble into liposomes in the presence of water and the mRNA.
d. Extrusion or Sonication: To further refine the liposome size and improve homogeneity, the liposome mixture can be extruded through porous membranes or subjected to sonication. Instead of using extrusion or sonication method, this Example includes using a MAGIC printing system to form mRNA-liposome.
e. Purification: The liposomes may be purified to remove any excess lipids, free mRNA, or other impurities. Example purification methods include gel filtration or dialysis.
f. Characterization: The liposomes may be analyzed to determine their size, stability, and encapsulation efficiency. Techniques like dynamic light scattering and transmission electron microscopy can be used for characterization.
g. Storage: The encapsulated mRNA in liposomes may be stored under appropriate conditions (e.g., low or subzero temperatures) to maintain stability until ready for use.

### Example 4—Additional Example Magic System Components

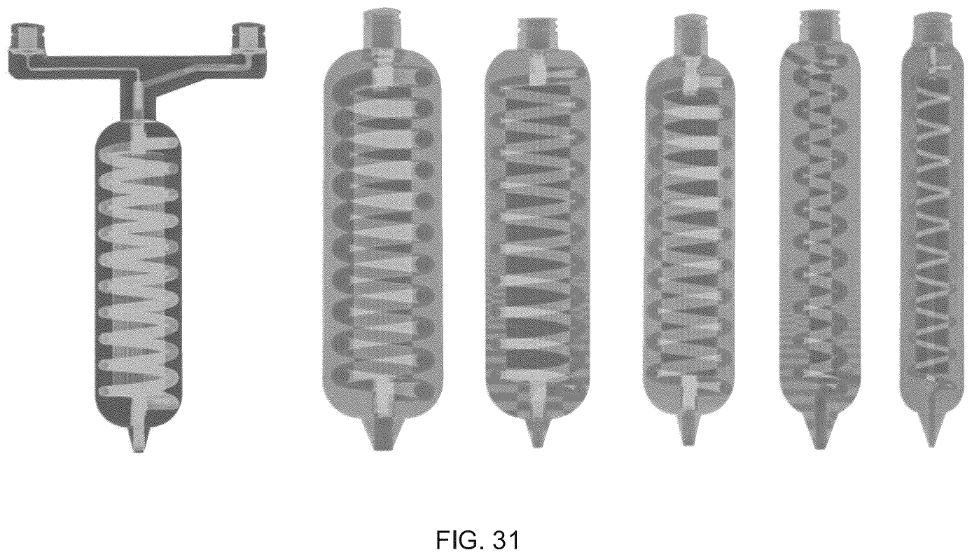

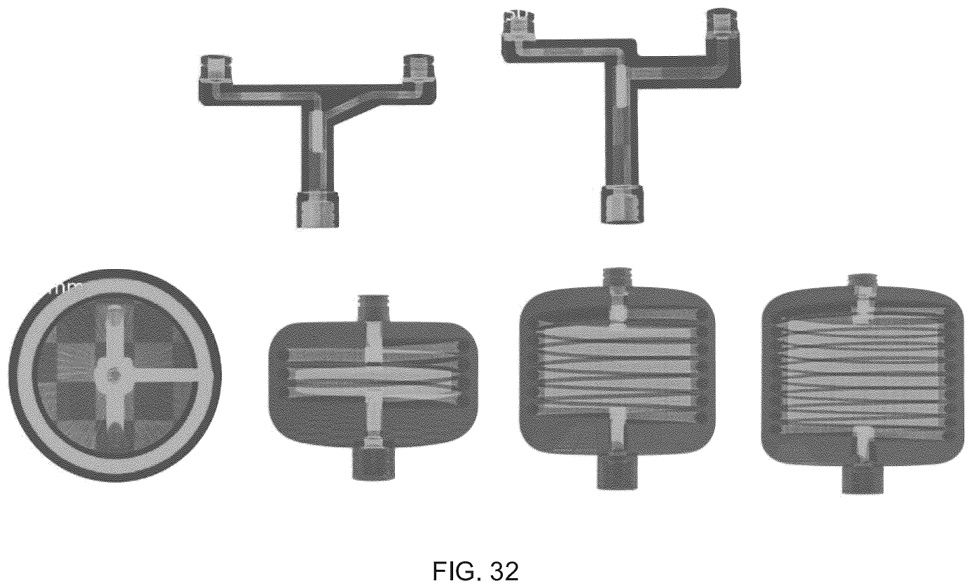

FIG. 30, FIG. 31, and FIG. 32 provide numerous views of example printheads and components. FIG. 30 depicts 3D models of several example linker systems, which may be used to provide for inlets for combining the multiple components in a printhead. FIG. 31 depicts 3D models of an example linker system coupled to a helical mixing system (left) as well as additional example helical mixing systems. FIG. 31 depicts 3D models of example linker systems (top) as well as additional example helical mixing systems.

### Statements Regarding Incorporation by Reference and Variations

All references throughout this application, for example, patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and sub-combinations possible of the group are intended to be individually included in the disclosure. As used herein, “and/or” means that one, all, or any combination of items in a list separated by “and/or” are included in the list; for example, “1, 2, and/or 3” is equivalent to “1, 2, 3, 1 and 2, 1 and 3, 2 and 3, or 1, 2 and 3”.

Every formulation or combination of components described or exemplified can be used to practice the invention unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resorting to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges, and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art and that such modifications and variations are considered to be within the scope of this invention as defined by the appended claims.

1. A method comprising:
providing a first component to a first linker of a plurality of linkers of a printhead;
providing a second component to a second linker of the plurality of linkers of the printhead;
contacting the first component and the second component in a fluid mixer of the printhead;
forming a mixture of the first component and the second component in the printhead; and
printing the mixture from the printhead through one or more outlets.

2. The method of claim 1, wherein the first or second component comprises a solvent, a polymer, a non-polymeric excipient, an active pharmaceutical ingredient, a protein, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, live mesenchymal stem cell, a biodegradable polymer, poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, fibroin, lipids, a polyethylene glycol (PEG) derivative, a pharmaceutical grade polymer, poly(hydroxy butyrate), poly(β-malic acid), or poly(L-lysine), a hydrophilic substance, a hydrophobic substance, a non-reducing sugar, trehalose, sucrose, a polyol, mannitol, sorbitol, xylitol, an amino acid, leucine, or L-arginine.

3. (canceled)

4. The method of claim 1, wherein contacting the first component and the second component comprises:
providing the first component to an inlet of the fluid mixer in the printhead; and
providing the second component to the inlet of the fluid mixer in the printhead.

5-6. (canceled)

7. The method of claim 1, wherein the fluid mixer comprises a stirrer, and the method further comprises using an electric field or a magnetic field to activate the stirrer.

8-12. (canceled)

13. The method of claim 1, wherein the printing comprises:
a) subjecting the mixture to shear forces that separate the mixture into a plurality of droplets including particles; or
b) using an extrusion-based printing method to generate a plurality of droplets including particles having diameters of from 10 nm to 1100 μm.

14. The method of claim 13, wherein the printing comprises subjecting the mixture to shear forces that separate the mixture into a plurality of droplets including particles, and wherein the method further comprises:
a) subjecting the droplets to evaporation conditions to evaporate the solvent from the droplets and leave the particles;
b) collecting the plurality of droplets, the collecting comprising receiving the plurality of droplets on a surface having a temperature of about room temperature, less than or about −78° C. or from about 200° C. to about room temperature; or
c) lyophilizing the plurality of droplets or the particles.

15. The method of claim 14, wherein the droplets are subjected to evaporation conditions to evaporate the solvent from the droplets and leave the particles, and further comprising washing the particles.

16. The method of claim 1, wherein the mixture comprises or further comprises one or more of a cosolvent, a surfactant, a preservative, live cells, cellular components, an additional active ingredient, a salt, a preservative, a protein, a peptide, an amino acid, or a nucleic acid component.

17-21. (canceled)

22. The method of claim 13, wherein the printing comprises using an extrusion-based printing method to generate a plurality of droplets including particles having diameters of from 10 nm to 1100 μm, and wherein the extrusion-based printing method
a) subjects the mixture to a pressure of greater than or about 200 kPA or from 10 kPa to 700 kPa; or
b) uses a nozzle having a diameter of from 100 μm to 1000 μm, from 100 μm to 700 μm, or from 300 μm to 700 μm.

23-27. (canceled)

28. A system comprising:
the printhead of claim 44;
a plurality of supply containers for preparing or storing a respective component comprising a polymer or a non-polymeric excipient, a solvent, or an active pharmaceutical ingredient;
wherein the printhead is in fluid communication with the plurality of supply containers;
and
a collection surface for receiving a plurality of droplets of the mixture from the one or more outlets.

29-31. (canceled)

32. The system of claim 28, further comprising at least one of
a) a cooling or refrigeration system coupled to the collection surface for cooling the collection surface to a temperature of from about 200° C. to about 75° C.;
b) one or more temperature sensors or temperature controllers for monitoring of controlling a temperature of the collection surface; or
c) one or more mixing vessels in fluid communication with the plurality of supply containers for preparing and providing components to the plurality of supply containers.

33-35. (canceled)

36. The system of claim 28, wherein the collection surface
a) is a moving or translating collection surface;
b) is cooled to a temperature of from about 200° C. to about 75° C.; or
c) comprises a sterile vial.

37. (canceled)

38. The system of claim 28, wherein the one or more outlets of the printhead comprise one or more extrusion-based printing nozzles for generating a plurality of droplets of the mixture including particles having diameters of from 10 nm to 1100 μm, and wherein the system further comprises at least one of
a) a translation stage for generating a relative translation between the one or more extrusion-based printing nozzles and the collection surface;
b) one or more actuators, pressure sensors or pressure controllers for monitoring or controlling an extrusion pressure or extrusion speed associated with the one or more extrusion-based printing nozzles;
c) a housing for maintaining at least the one or more extrusion-based printing nozzles and the collection surface in a sterile environment; or
d) sterilization equipment positioned to sterilize one or more of the plurality of supply containers, the one or more extrusion-based printing nozzles, or the collection surface.

39-43. (canceled)

44. A printhead comprising:
a plurality of linkers defining inlets of the printhead;
a fluid mixer in fluid communication with the plurality of linkers for mixing components from the inlets into a mixture; and
one or more outlets in fluid communication with the fluid mixer.

45. The printhead of claim 44, wherein one or more of the plurality of linkers
a) comprise a Luer lock, a Luer slip, or a slip tip;
b) are characterized by a diameter of less than or about 5.0 mm; or
c) are coupleable to an outlet of an additional printhead.

46. (canceled)

47. The printhead of claim 44, wherein the fluid mixer
a) comprises a coaxial arrangement of a first fluidic channel in fluid communication with a first linker and a second fluid channel in fluid communication with a second linker;
b) is characterized by a fluidic arrangement providing flows of components from the plurality of linkers in a coaxial flow configuration, in a biaxial flow configuration, or in a triaxial or higher order flow configuration;
c) comprises a stirrer;
d) comprises a mixing architecture characterized by an “S” shape or a helix shape; or
e) is in contact with one or more temperature sensors or temperature controllers for monitoring or controlling a temperature of the fluid mixture.

48-49. (canceled)

50. The printhead of claim 47, wherein the fluid mixer comprises a stirrer, and
a) the printhead further comprises an electric field or a magnetic field for activating the stirrer; or
b) the stirrer rotates about an axis parallel to a flow direction of the mixture through the one or more outlets or rotates about an axis perpendicular to a flow direction of the mixture through the one or more outlets.

51-56. (canceled)

57. The printhead of claim 44, wherein the one or more outlets
a) are characterized by a diameter of less than or about 5.0 mm; or
b) comprise one or more extrusion-based printing nozzles for generating a plurality of droplets of the mixture including particles having diameters of from 10 nm to 1100 μm.

58. (canceled)

59. A method comprising:
manufacturing a printhead, wherein the printhead comprises:
a plurality of linkers defining inlets of the printhead;
a fluid mixer in fluid communication with the plurality of linkers for mixing components from the inlets into a mixture; and
one or more outlets in fluid communication with the fluid mixer.

60. The method of claim 59, wherein manufacturing the printhead comprises additive manufacturing.

61. The method of claim 59, further comprising
a) 3D modelling the printhead prior to manufacturing the printhead;
b) designating a 3D model for the printhead; or
c) modeling flow and mixing within the fluid mixer prior to manufacturing the printhead.

62-63. (canceled)
